(19) United States
(12) Patent Application Publication
(10) Pub. No.: US 2025/0000720 A1
(43) Pub. Date: Jan. 2, 2025

(54) ABSORBENT CORES AND METHODS OF MANUFACTURING

(71) Applicant: Kimberly-Clark Worldwide, Inc., Neenah, WI (US)

(72) Inventors: Man Li, Beijing (CN); Bradley W. Schoon, Oshkosh, WI (US); Thomas G. Vercauteren, Appleton, WI (US); Daniel T. Labash, Appleton, WI (US); Mark J. Beitz, Appleton, WI (US); TongTong Zhang, Beijing (CN); Jing Shi, Shanghai (CN); Jue Wang, Shanghai (CN); Andrew T. Baker, Norcross, GA (US)

(21) Appl. No.: 18/697,840

(22) PCT Filed: Oct. 25, 2021
(86) PCT No.: PCT/CN2021/125981

§ 371 Date: Apr. 2, 2024

(51) Int. Cl. A61F 13/538; A61F 13/53; A61F 13/539

(52) U.S. Cl. CPC A61F 13/538; A61F 13/539; A61F 2013/530481; A61F 2013/53908

(57) ABSTRACT

Absorbent structures and methods of forming absorbent structures are disclosed. An absorbent structure may comprise a body side liner (28), an outer cover (26), and an absorbent structure. The absorbent structure may comprise a top material, a bottom material, a lofty nonwoven reinforcing material between the top material and the bottom material, and an absorbent layer of superabsorbent particles (318) and adhesive filaments (316) between the top material and the lofty nonwoven. The superabsorbent particles (318) may be present at greater than 90% by weight of absorbent material, and the adhesive filaments (316) may form a three-dimensional mesh network comprising network adhesive filaments (316) with the superabsorbent particles (318) immobilized within the mesh network, the network adhesive filaments (316) and superabsorbent particles (318) extending throughout a three-dimensional space defined by the network adhesive filaments (316) and the superabsorbent particles (318), with the network adhesive filaments (316) extending in random orientations throughout the three-dimensional space.

### TECHNICAL FIELD

The present disclosure is directed to absorbent cores and more specifically to absorbent cores with a high superabsorbent material content.

### BACKGROUND OF THE DISCLOSURE

A primary function of personal care absorbent articles is to absorb and retain body exudates such as urine, fecal material, blood, and menses with additional desired attributes including low leakage of the exudates from the absorbent article and a dry feel to the wearer of the absorbent article. By preventing leakage of the exudates from the absorbent article, the absorbent article intends to prevent the body exudates from soiling or contaminating a wearer's or caregiver's clothing or other articles, such as bedding, that can come in contact with the wearer.

Absorbent cores typically help with liquid uptake and storage within absorbent articles. Many absorbent cores contain multiple absorbent materials such as superabsorbent material and pulp fluff or other fibrous absorbent material. Each type of absorbent material helps to impart such absorbent cores with a range of properties useful in absorbing and retaining liquid bodily exudates. For example, pulp fluff or other fibrous absorbent material may absorb liquid more quickly than superabsorbent material, and the superabsorbent material may be able retain more liquid per particle than pulp fluff.

Many advances have been made to absorbent cores, and particularly to the superabsorbent material of absorbent cores. Some current absorbent cores may now have absorbent material comprising mostly superabsorbent material and further comprising only a small portion of other absorbent material. Other current absorbent cores comprise only superabsorbent material as the absorbent material. For example, FIG. 6 represents a cross-section of a known, exemplary absorbent structure 1030. As shown, the absorbent structure 1030 includes a top facing material layer 1042, which may be disposed proximate a bodyside liner within an absorbent article. The absorbent structure 1030 further includes a bottom facing material layer 1044, which may be disposed proximate an outer cover within an absorbent article. The absorbent structure 1030 further includes a first adhesive layer 1062 and a second adhesive layer 1064, with a first absorbent layer 1063 disposed between the first adhesive layer 1062 and the second adhesive layer 1064. The first absorbent layer 1062 comprises superabsorbent particles 1055.

The absorbent structure 1030 includes a third adhesive layer 1054 and a fourth adhesive layer 1052, with a second absorbent layer 1053 disposed between the third adhesive layer 1054 and the fourth adhesive layer 1052. The second absorbent layer 1053 comprises superabsorbent particles 1055. Between the first absorbent layer 1063 and the second absorbent layer 1053 is an internal reinforcing material 1046. Finally, the absorbent core 1030 includes end seal adhesive 1067 disposed on transverse sides of the structure 1030.

Such constructions as exemplary absorbent structure 1030 can be made to be thinner and more flexible than some absorbent bodies which contain traditional pulp fluff absorbent material. However, further improvements to such pulp-free absorbent bodies are a continued area of exploration to increase their performance.

### SUMMARY OF THE DISCLOSURE

In one embodiment, an absorbent article may extend in a longitudinal and a lateral direction and comprise an inner bodyside liner; an outer cover; and an absorbent structure disposed between the inner bodyside liner and the outer cover. The absorbent structure may comprise a top facing material layer disposed adjacent the bodyside liner; a bottom facing material layer disposed adjacent the outer cover; a lofty nonwoven reinforcing material disposed between the top facing material layer and the bottom facing material layer; and a first absorbent layer comprising superabsorbent particles intermixed with adhesive filaments disposed between the top facing material layer and the lofty nonwoven reinforcing material or between the lofty nonwoven reinforcing material and the bottom facing material layer. The superabsorbent particles may be present in an amount greater than or equal to 90%, by weight of absorbent material between the top facing material layer and the bottom facing material layer, and the adhesive filaments may form a three-dimensional mesh network comprising network adhesive filaments with the superabsorbent particles immobilized within the mesh network, the network adhesive filaments and superabsorbent particles extending throughout a three-dimensional space defined by the network adhesive filaments and the superabsorbent particles, with the network adhesive filaments extending in random orientations throughout the three-dimensional space.

In another embodiment, an absorbent article may extend in a longitudinal and a lateral direction and comprise: an inner bodyside liner; an outer cover; and an absorbent structure disposed between the inner bodyside liner and the outer cover. The absorbent structure may comprise a top facing material layer disposed adjacent the bodyside liner; a bottom facing material layer disposed adjacent the outer cover; a lofty nonwoven reinforcing material disposed between the top facing material layer and the bottom facing material layer; a first absorbent layer comprising superabsorbent particles intermixed with adhesive filaments disposed between the top facing material layer and the lofty nonwoven reinforcing material, the superabsorbent particles of the first absorbent layer being present in an amount between 100 gsm and 250 gsm; and a second absorbent layer comprising superabsorbent particles intermixed with adhesive filaments disposed between the lofty nonwoven reinforcing material the bottom facing material layer, the superabsorbent particles of the second absorbent layer being present in an amount between 100 gsm and 250 gsm. The superabsorbent particles may be present in an amount greater than or equal to 90%, by weight of absorbent material between the top facing material layer and the bottom facing material layer, wherein a total quantity of adhesive disposed between the top facing material layer and the bottom facing material layer may be present in an amount less than 4.5%, by weight of the superabsorbent particles present between the top facing material layer and the bottom facing material layer, and wherein the adhesive filaments may form a three-dimensional mesh network comprising network adhesive filaments with the superabsorbent particles immobilized within the mesh network, the network adhesive filaments and superabsorbent particles extending throughout a three-dimensional space defined by the network adhesive filaments and the superabsorbent particles, with the network adhesive filaments extending in random orientations throughout the three-dimensional space.

In a further embodiment, an absorbent article may extend in a longitudinal and a lateral direction and comprise an inner bodyside liner; an outer cover; and an absorbent structure disposed between the inner bodyside liner and the outer cover comprising superabsorbent particles present in an amount greater than or equal to 90%, by weight of absorbent material of the absorbent structure. The absorbent structure may further comprise a top facing material layer disposed adjacent the bodyside liner; a bottom facing material layer disposed adjacent the outer cover; a lofty nonwoven reinforcing material disposed between the top facing material layer and the bottom facing material layer; a first absorbent layer comprising superabsorbent particles intermixed with adhesive filaments disposed between the top facing material layer and the lofty nonwoven reinforcing material, the superabsorbent particles of the first absorbent layer being present in an amount greater than 0 gsm and less than 200 gsm; and a second absorbent layer comprising superabsorbent particles intermixed with adhesive filaments disposed between the lofty nonwoven reinforcing material the bottom facing material layer, the superabsorbent particles of the second absorbent layer being present in an amount greater than 0 gsm and less than 200 gsm. A total quantity of adhesive disposed between the top facing material layer and the bottom facing material layer may be present in an amount less than 4.0%, by weight of the superabsorbent particles present between the top facing material layer and the bottom facing material layer, wherein the adhesive filaments may form a three-dimensional mesh network comprising network adhesive filaments with the superabsorbent particles immobilized within the mesh network, the network adhesive filaments and superabsorbent particles extending throughout a three-dimensional space defined by the network adhesive filaments and the superabsorbent particles, with the network adhesive filaments extending in random orientations throughout the three-dimensional space, and wherein a SAM penetration value of the absorbent structure is less than or equal to 65%, according to the SAM Penetration Test Method.

### BRIEF DESCRIPTION OF DRAWINGS

A full and enabling disclosure thereof, directed to one of ordinary skill in the art, is set forth more particularly in the remainder of the specification, which makes reference to the appended figures in which:

FIG. 1 is side perspective view of an exemplary embodiment of an absorbent article, such as a diaper, in a fastened condition.

FIG. 2 is a top plan view of the absorbent article of FIG. 1 in a stretched, laid flat, unfastened condition.

FIG. 3 is a front perspective view of an alternative embodiment of an absorbent article, such as a pant.

FIG. 4 is a top plan view of the absorbent article of FIG. 3 in a stretched, laid flat condition.

FIG. 5 is a front perspective cross-sectional view taken along line 5-5 from FIG. 2, with the absorbent article being in a relaxed configuration.

FIG. 6 is a cross-section view of an exemplary prior art absorbent core.

FIG. 7 is a process schematic depicting an exemplary method of manufacturing an absorbent core according to the present disclosure.

FIG. 8 is a process schematic depicting a portion of the exemplary method of FIG. 6.

FIG. 9 is a process schematic depicting an alternative exemplary method of manufacturing an absorbent structure according to the present disclosure.

FIG. 10A-10C are exemplary front cross-sectional views of an absorbent structure formed according to a method of manufacturing according to aspects of the present disclosure, taken along line 10-10 from FIG. 7.

FIG. 11A-11C are alternative exemplary front cross-sectional views of an absorbent structure formed according to a method of manufacturing according to aspects of the present disclosure, taken along line 11-11 from FIG. 9.

FIG. 12A is a top perspective view of a three-dimensional image generated by a micro-CT process used to analyze an exemplary mixture of particles and adhesive filaments formed by the process of FIG. 9, according to aspects of the present disclosure.

FIG. 12B is a top plan view of the three-dimensional image of FIG. 12A.

FIG. 12C is a cross-sectional view of a slice of the three-dimensional image of FIG. 12A.

FIG. 12D is the cross-sectional view of FIG. 12C with the particles leaving only the adhesive filaments.

Repeat use of reference characters in the present specification and drawings is intended to represent the same or analogous features or elements of the disclosure.

### DETAILED DESCRIPTION OF THE DISCLOSURE

In an embodiment, the present disclosure is generally directed towards absorbent cores absorbent material comprising a high proportion of superabsorbent material. Each example is provided by way of explanation and is not meant as a limitation. For example, features illustrated or described as part of one embodiment or figure can be used on another embodiment or figure to yield yet another embodiment. It is intended that the present disclosure include such modifications and variations.

When introducing elements of the present disclosure or the preferred embodiment(s) thereof, the articles “a”. “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products and other adult care garments, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” or “coupled” refers herein to the joining, adhering, connecting, attaching, or the like, of two elements. Two elements will be considered bonded or coupled together when they are joined, adhered, connected, attached, or the like, directly to one another or indirectly to one another, such as when each is directly bonded to intermediate elements. The bonding or coupling of one element to another can occur via continuous or intermittent bonds.

The term “carded web” refers herein to a web containing natural or synthetic staple length fibers typically having fiber lengths less than 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

“Elastomeric” refers to a material or composite which can be elongated by at least 50 percent of its relaxed length and which will recover, upon release of the applied force, at least 20 percent of its elongation. It is generally preferred that the elastomeric material or composite be capable of being elongated by at least 50 percent, more preferably by at least 100 percent, and still more preferably by at least 300 percent of its relaxed length and recover, upon release of an applied force, at least 50 percent of its elongation.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than 0.3, and in an embodiment, between 0.6, 5 and 10 and 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least 15 times its weight and, in an embodiment, at least 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “super-majority” refers herein to a majority of at least 65%.

The term “thermoplastic” refers herein to a material which softens and which can be shaped when exposed to heat and which substantially returns to a non-softened condition when cooled.

The term “user” or “caregiver” refers herein to one who fits an absorbent article, such as, but not limited to, a diaper, diaper pant, training pant, youth pant, incontinent product, or other absorbent article about the wearer of one of these absorbent articles. A user and a wearer can be one and the same person.

### Absorbent Article:

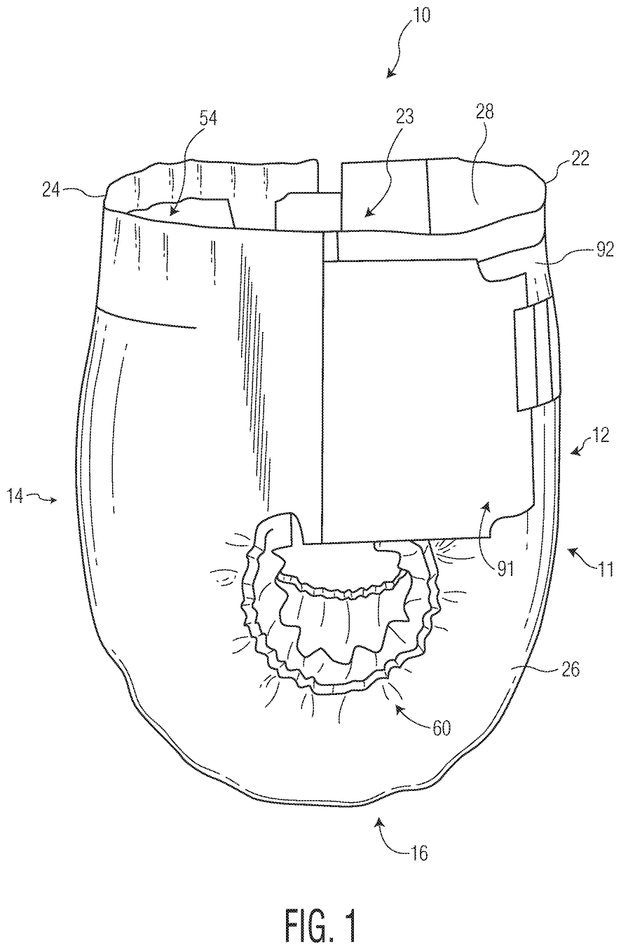

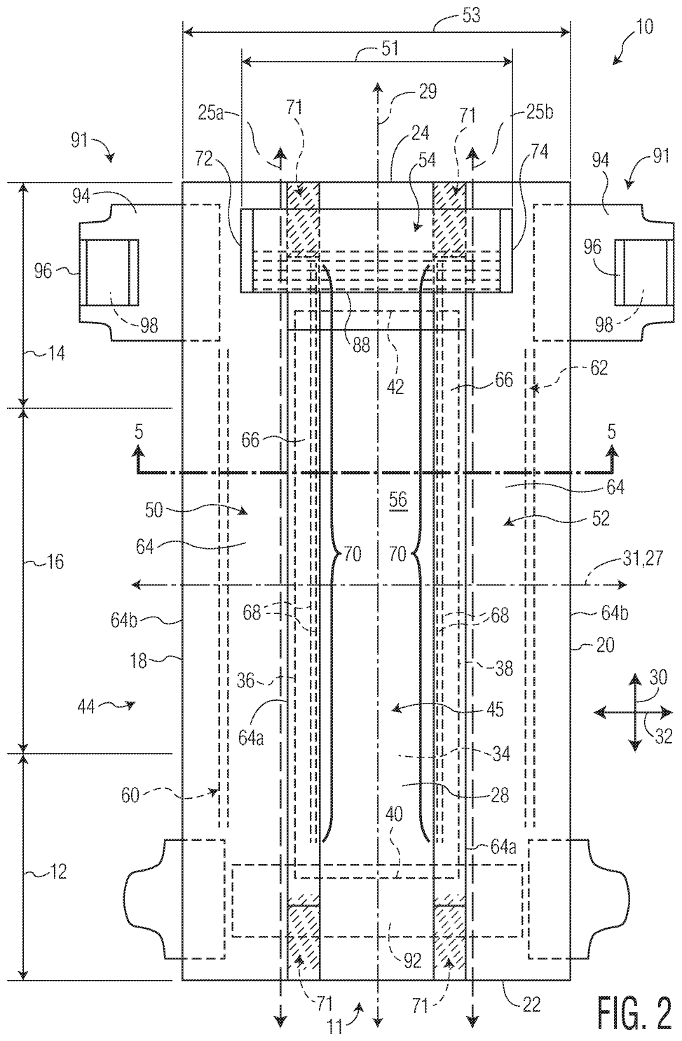

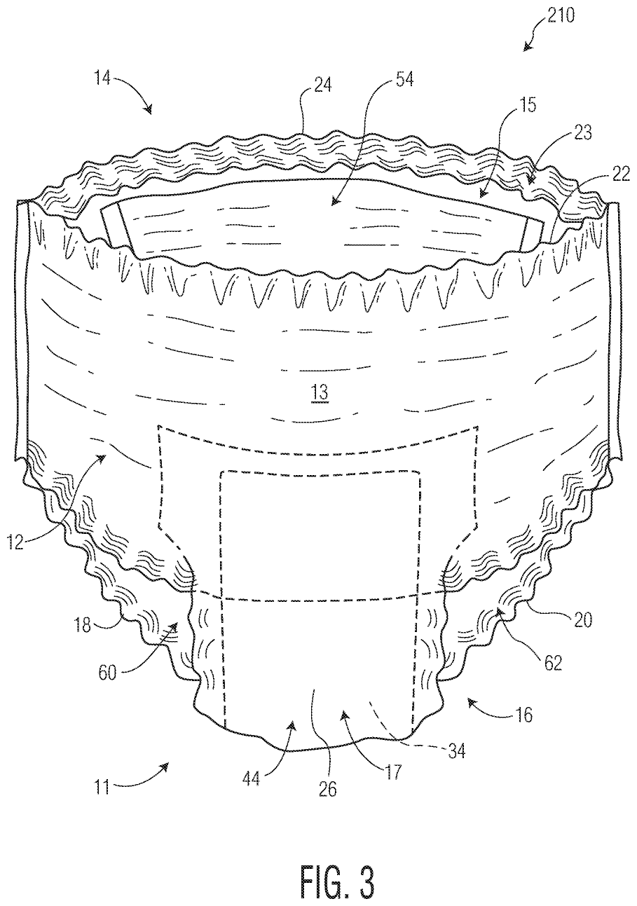

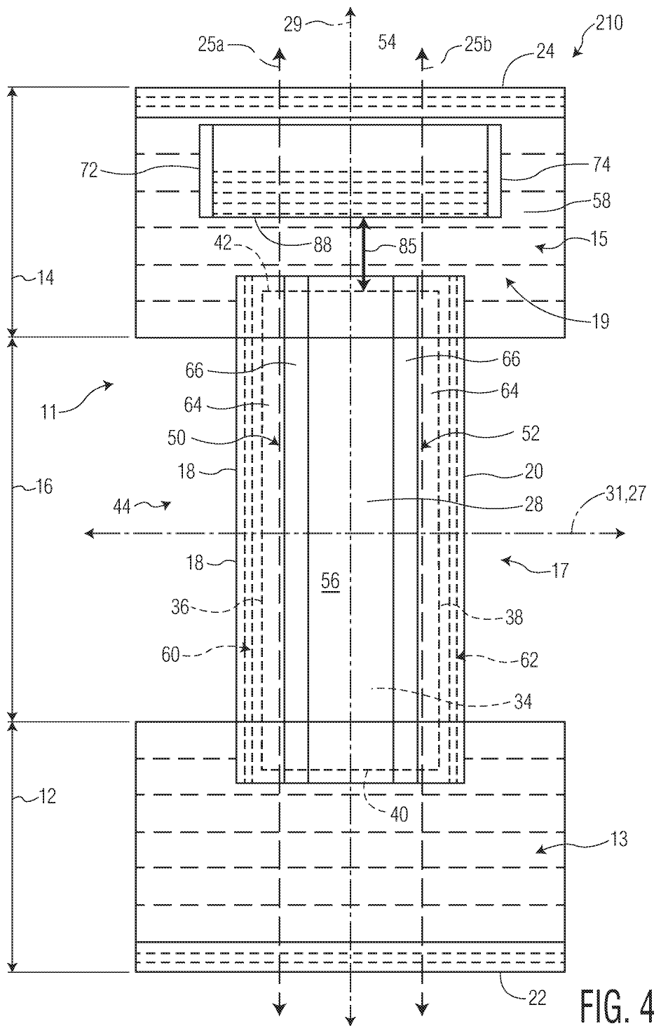

Referring to FIGS. 1-2, a non-limiting illustration of an absorbent article 10, for example a diaper, is illustrated. While the examples and illustrations described herein may generally apply to absorbent articles manufactured in the product longitudinal direction, which is hereinafter called the machine direction manufacturing of a product, it should be noted that one of ordinary skill in the art could apply the information herein to absorbent articles manufactured in the latitudinal direction of the product, which hereinafter is called the cross-machine direction manufacturing of a product, without departing from the spirit and scope of the disclosure. For example, the absorbent article 210 in FIGS. 3-4 provide an exemplary absorbent article 210 that can be manufactured in cross-machine direction manufacturing process.

The absorbent article 10 illustrated in FIGS. 1 and 2 and the absorbent article 210 illustrated in FIGS. 3 and 4 each generally comprise a chassis 11. The absorbent article 10, 210 has a front waist region 12, a rear waist region 14, and a crotch region 16 disposed between the front waist region 12 and the rear waist region 14 and interconnecting the front and rear waist regions, 12, 14, respectively. The front waist region 12 may be alternatively referred to as the front-end region, the rear waist region 14 as the rear-end region, and the crotch region 16 as the intermediate region. In the examples of FIGS. 3 and 4, a three-piece construction of an absorbent article 210 is depicted where the absorbent article 210 has a chassis 11 including a front waist panel 13 defining the front waist region 12, a rear waist panel 15 defining the rear waist region 14, and an absorbent panel 17 defining the crotch region 16 of the absorbent article 210. The absorbent panel 17 of FIGS. 3 and 4 extends between the front waist panel 13 and the rear waist panel 15. The absorbent panel 17 can overlap the front waist panel 13 and the rear waist panel 15 and be bonded to the front waist panel 13 and the rear waist panel 15 to define a three-piece construction. However, it is contemplated that an absorbent article can be manufactured in a cross-machine direction without being a three-piece construction garment—for example, constructed with a one-piece outer cover.

The absorbent article 10, 210 has a pair of longitudinal side edges 18, 20, and a pair of opposite waist edges, respectively designated front waist edge 22 and rear waist edge 24. The front waist region 12 can be contiguous with the front waist edge 22 and the rear waist region 14 can be contiguous with the rear waist edge 24. The longitudinal side edges 18, 20 can extend from the front waist edge 22 to the rear waist edge 24. The longitudinal side edges 18, 20 can extend in a direction parallel to the longitudinal direction 30 for their entire length, such as for the absorbent article 10 illustrated in FIGS. 1 and 2. In other embodiments, the longitudinal side edges 18, 20 can be curved between the front waist edge 22 and the rear waist edge 24. In the absorbent article 210 of FIGS. 3 and 4, the longitudinal side edges 18, 20 can include portions of the front waist panel 13, the absorbent panel 17, and the rear waist panel 15.

The front waist region 12 may generally correspond to the portion of the absorbent article 10, 210 that, when worn, is positioned at least in part on the front of the wearer while the rear waist region 14 may generally correspond to the portion of the absorbent article 10, 210 that, when worn, is positioned at least in part on the back of the wearer. The crotch region 16 of the absorbent article 10, 210 is disposed between the front waist region 12 and the rear waist region 14 and is the portion of the absorbent article 10, 210 that, when worn, is positioned between the legs of the wearer and can partially cover the lower torso of the wearer. The waist edges, 22 and 24, of the absorbent article 10, 210 are configured to encircle the waist of the wearer and together define a central waist opening 23 (as labeled in FIG. 1 and FIG. 3) for the waist of the wearer. Portions of the longitudinal side edges 18, 20 in the crotch region 16 can generally define leg openings for the legs of the wearer when the absorbent article 10, 210 is worn.

The example absorbent article 10, 210 of FIGS. 1-4 includes an outer cover 26 and a bodyside liner 28. The outer cover 26 and the bodyside liner 28 can form a portion of the chassis 11. In an example of absorbent articles contemplated by the present disclosure, the bodyside liner 28 can be bonded to the outer cover 26 in a superposed relation by any suitable means such as, but not limited to, adhesives, ultrasonic bonds, thermal bonds, pressure bonds, or other conventional techniques. The outer cover 26 defines a length in a longitudinal direction 30, and a width in the lateral direction 32, which, in the illustrated example of article 10 in FIGS. 1-2, can coincide with the length and width of the absorbent article 10. As illustrated in FIGS. 2 and 4, the absorbent article 10, 210 has a longitudinal axis 29 extending in the longitudinal direction 30 and a lateral axis 31 extending in the lateral direction 32.

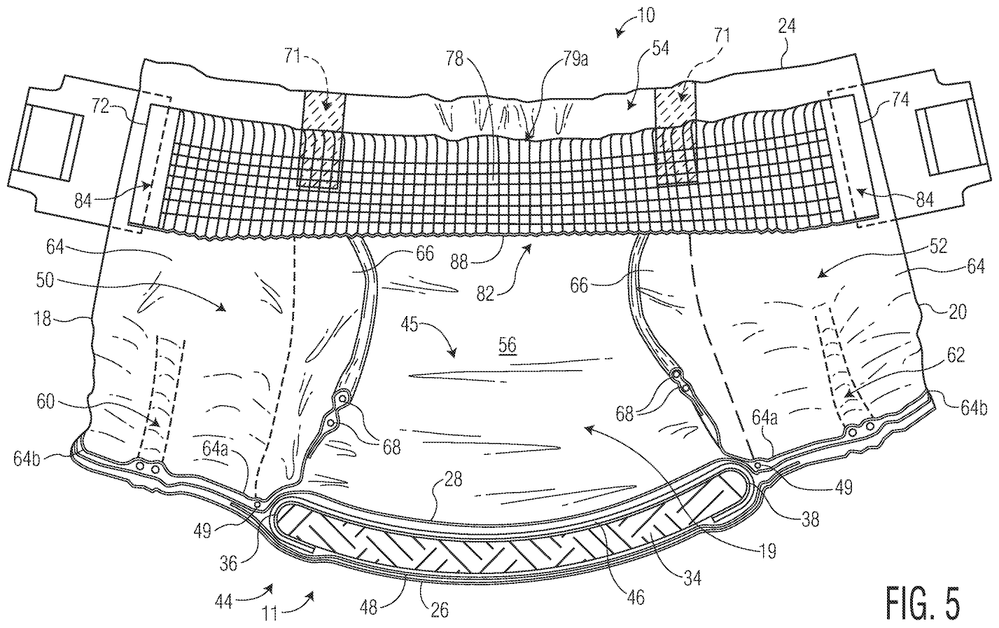

The chassis 11 includes an absorbent core 34. The absorbent core 34 is generally disposed between the outer cover 26 and the bodyside liner 28. The absorbent core 34 has longitudinal edges, 36 and 38, which, in at least some examples, can form portions of the longitudinal side edges, 18 and 20, respectively, of the absorbent article 10, 210. The absorbent core 34 has a first end edge 40 that is opposite a second end edge 42, respectively, which, in particular examples, form portions of the waist edges, 22 and 24, respectively, of the absorbent article 10. In some examples of the article 10, 210, the first end edge 40 is in the front waist region 12 and/or the second end edge 42 is in the rear waist region 14. In some examples of the article 10, 210, the absorbent core 34 has a length and width that are the same as or less than the length and width of the absorbent article 10, 210. The bodyside liner 28, the outer cover 26, and the absorbent core 34 can be considered to form part of an absorbent assembly 44 in some examples. In the absorbent article 210 of FIGS. 3 and 4, the absorbent panel 17 can form the absorbent assembly 44. In some examples of the article 10, 210, the absorbent assembly 44 can further include a fluid transfer layer 46 (as shown in FIG. 5) and/or a fluid acquisition layer (not shown) between the bodyside liner 28 and the fluid transfer layer 46 as is known in the art. The absorbent assembly 44 may also include a spacer layer 48 (as shown in FIG. 5) disposed between the absorbent core 34 and the outer cover 26.

The absorbent article 10, 210 are generally configured to contain and/or absorb liquid, solid, and semi-solid body exudates discharged from the wearer. In some examples, containment flaps 50, 52 can be configured to provide a barrier to the lateral flow of body exudates. To further enhance containment and/or absorption of body exudates, the absorbent article 10, 210 can suitably include a waist containment member 54. In some embodiments, the waist containment member 54 can be disposed in the rear waist region 14 of the absorbent article 10, 210. Although not depicted herein, it is contemplated that the waist containment member 54 can be additionally or alternatively disposed in the front waist region 12 of the absorbent article 10, 210.

The waist containment member 54 can be disposed on the body facing surface 19 of the chassis 11 to help contain and/or absorb body exudates. In some embodiments, such as in the absorbent articles 10 depicted in FIGS. 1 and 2, the waist containment member 54 can be disposed on the body facing surface 45 of the absorbent assembly 44. In some embodiments, the waist containment member 54 can be disposed on the body facing surface 56 of the bodyside liner 28. In some embodiments, such as in the absorbent article 210 depicted in FIGS. 3 and 4, the waist containment member 54 can be disposed on the body facing surface 58 of the rear waist panel 15.

The absorbent article 10, 210 can further include leg elastic members 60, 62 as are known to those skilled in the art. The leg elastic members 60, 62 can be attached to the outer cover 26 and/or the bodyside liner 28 along the opposite longitudinal side edges. 18 and 20, and positioned in the crotch region 16 of the absorbent article 10, 210. The leg elastic members 60, 62 can be parallel to the longitudinal axis 29 as shown in FIGS. 2 and 4 or can be curved as is known in the art. The leg elastic members 60, 62 can be elastomeric and can provide elasticized leg cuffs.

In some embodiments, the absorbent article 10, 210 can further include longitudinal extending fold lines 25a, 25b, as shown in FIGS. 2 and 4. The first longitudinal extending fold line 25a can be on one side of the longitudinal axis 29 of the absorbent article 10, 210 and the second longitudinal extending fold line 25b can be on an opposite side of the longitudinal axis 29. In some embodiments, the longitudinal extending fold lines 25a, 25b can be generally parallel to the longitudinal axis 29 of the absorbent article 10, 210. In some embodiments, the absorbent article 10, 210 can further include a lateral extending fold line 27. The lateral extending fold line 27 can be parallel to and located at the lateral axis 31 of the absorbent article 10, 210 in some embodiments.

Additional details regarding each of these elements of the absorbent article 10, 210 described herein can be found below and with reference to the Figures.

### Outer Cover:

The outer cover 26 and/or portions thereof can be breathable and/or liquid impermeable. The outer cover 26 and/or portions thereof can be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer cover 26 can be constructed of a microporous polymeric film, such as polyethylene or polypropylene.

In an embodiment, the outer cover 26 can be a single layer of a liquid impermeable material, such as a polymeric film. In an embodiment, the outer cover 26 can be suitably stretchable, and more suitably elastic, in at least the lateral direction 32 of the absorbent article 10, 210. In an embodiment, the outer cover 26 can be stretchable, and more suitably elastic, in both the lateral 32 and the longitudinal 30 directions. In an embodiment, the outer cover 26 can be a multi-layered laminate in which at least one of the layers is liquid impermeable. In some embodiments, the outer cover 26 can be a two-layer construction, including an outer layer (not shown) and an inner layer (not shown) which can be bonded together such as by a laminate adhesive. Suitable laminate adhesives can be applied continuously or intermittently as beads, a spray, parallel swirls, or the like, but it is to be understood that the inner layer can be bonded to the outer layer by other bonding methods, including, but not limited to, ultrasonic bonds, thermal bonds, pressure bonds, or the like.

The outer layer of the outer cover 26 can be any suitable material and may be one that provides a generally cloth-like texture or appearance to the wearer. An example of such material can be a 100% polypropylene bonded-carded web with a diamond bond pattern available from Sandler A. G., Germany, such as 30 gsm Sawabond 4185@ or equivalent. Another example of material suitable for use as an outer layer of an outer cover 26 can be a 20 gsm spunbond polypropylene non-woven web. The outer layer may also be constructed of the same materials from which the bodyside liner 28 can be constructed as described herein.

The liquid impermeable inner layer of the outer cover 26 (or the liquid impermeable outer cover 26 where the outer cover 26 is of a single-layer construction) can be either vapor permeable (i.e., “breathable”) or vapor impermeable. The liquid impermeable inner layer (or the liquid impermeable outer cover 26 where the outer cover 26 is of a single-layer construction) can be manufactured from a thin plastic film. The liquid impermeable inner layer (or the liquid impermeable outer cover 26 where the outer cover 26 is of a single-layer construction) can inhibit liquid body exudates from leaking out of the absorbent article 10, 210 and wetting articles, such as bed sheets and clothing, as well as the wearer and caregiver.

In some embodiments, where the outer cover 26 is of a single layer construction, it can be embossed and/or matte finished to provide a more cloth-like texture or appearance. The outer cover 26 can permit vapors to escape from the absorbent article 10 while preventing liquids from passing through. A suitable liquid impermeable, vapor permeable material can be composed of a microporous polymer film or a non-woven material which has been coated or otherwise treated to impart a desired level of liquid impermeability.

### Bodyside Liner:

The bodyside liner 28 of the absorbent article 10, 110, 210 can overlay the absorbent core 34 and the outer cover 26 and can isolate the wearer's skin from liquid waste retained by the absorbent core 34. In various embodiments, a fluid transfer layer 46 can be positioned between the bodyside liner 28 and the absorbent core 34. In various embodiments, an acquisition layer (not shown) can be positioned between the bodyside liner 28 and the absorbent core 34 or a fluid transfer layer 46, if present. In various embodiments, the bodyside liner 28 can be bonded to the acquisition layer, or to the fluid transfer layer 46 if no acquisition layer is present, via adhesive and/or by a point fusion bonding. The point fusion bonding may be selected from ultrasonic, thermal, pressure bonding, and combinations thereof.

In an embodiment, the bodyside liner 28 can extend beyond the absorbent core 34 and/or a fluid transfer layer 46, if present, and/or an acquisition layer, if present, and/or a spacer layer 48, if present, to overlay a portion of the outer cover 26 and can be bonded thereto by any method deemed suitable, such as, for example, by being bonded thereto by adhesive, to substantially enclose the absorbent core 34 between the outer cover 26 and the bodyside liner 28. The bodyside liner 28 may be narrower than the outer cover 26. However, in other embodiments, the bodyside liner 28 and the outer cover 26 may be of the same dimensions in width and length. In other embodiments, the bodyside liner 28 can be of greater width than the outer cover 26. It is also contemplated that the bodyside liner 28 may not extend beyond the absorbent core 34 and/or may not be secured to the outer cover 26. In some embodiments, the bodyside liner 28 can wrap at least a portion of the absorbent core 34, including wrapping around both longitudinal edges 36, 38 of the absorbent core 34, and/or one or more of the end edges 40, 42. It is further contemplated that the bodyside liner 28 may be composed of more than one segment of material. The bodyside liner 28 can be of different shapes, including rectangular, hourglass, or any other shape. The bodyside liner 28 can be suitably compliant, soft feeling, and non-irritating to the wearer's skin and can be the same as or less hydrophilic than the absorbent core 34 to permit body exudates to readily penetrate through to the absorbent core 34 and provide a relatively dry surface to the wearer.

The bodyside liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 28. The bodyside liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, can include more than one layer of fabrics, films, and/or webs, as well as combinations thereof. For example, the bodyside liner 28 can include a support layer and a projection layer that can be hydroentagled. The projection layer can include hollow projections, such as those disclosed in U.S. Pat. No. 9,474,660 to Kirby, Scott S. C. et al.

For example, the bodyside liner 28 can be composed of a meltblown or spunbond web of polyolefin fibers. Alternatively, the bodyside liner 28 can be a bonded-carded web composed of natural and/or synthetic fibers. The bodyside liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire bodyside liner 28 or it can be selectively applied to particular sections of the bodyside liner 28.

In an embodiment, a bodyside liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a bodyside liner 28 can be a spunbond substrate with a basis weight from 10 or 12 to 15 or 20 gsm. In an embodiment, a bodyside liner 28 can be a 12 gsm spunbond-meltblown-spunbond substrate having 10% meltblown content applied between the two spunbond layers.

Although the outer cover 26 and bodyside liner 28 can include elastomeric materials, it is contemplated that the outer cover 26 and the bodyside liner 28 can be composed of materials which are generally non-elastomeric. In an embodiment, the bodyside liner 28 can be stretchable, and more suitably elastic. In an embodiment, the bodyside liner 28 can be suitably stretchable and more suitably elastic in at least the lateral or circumferential direction of the absorbent article 10, 210. In other aspects, the bodyside liner 28 can be stretchable, and more suitably elastic, in both the lateral and the longitudinal directions 32, 30, respectively.

### Containment Flaps:

In an embodiment, the absorbent article 10, 210 can include a pair of containment flaps 50, 52. The containment flaps 50, 52 can be formed separately from the absorbent chassis 11 and attached to the chassis 11 or can be formed integral to the chassis 11. In some embodiments, the containment flaps 50, 52 can be secured to the chassis 11 of the absorbent article 10, 210 in a generally parallel, spaced relation with each other laterally inward of the leg openings to provide a barrier against the flow of body exudates. One containment flap 50 can be on a first side of the longitudinal axis 29 and the other containment flap 52 can be on a second side of the longitudinal axis 29. In an embodiment, the containment flaps 50, 52 can extend generally in a longitudinal direction 30 from the front waist region 12 of the absorbent article 10, through the crotch region 16 to the rear waist region 14 of the absorbent article 10. In some embodiments, the containment flaps 50, 52 can extend in a direction substantially parallel to the longitudinal axis 29 of the absorbent article 10, 210, however, in other embodiments, the containment flaps 50, 52 can be curved, as is known in the art. In other embodiments, such as the absorbent article 210 in FIGS. 3 and 4, the containment flaps 50, 52 can be disposed on the absorbent panel 17 in the crotch region 16.

In embodiments where the containment flaps 50, 52 are coupled to the chassis 11, the containment flaps 50, 52 can be bonded to the bodyside liner 28 with a barrier adhesive 49, as shown in FIG. 5. Alternatively, the containment flaps 50, 52 can be bonded to the outer cover 26 with a barrier adhesive 49, or to the spacer layer 48. Of course, the containment flaps 50, 52 can be bonded to other components of the chassis 11 and can be bonded with other suitable means other than a barrier adhesive 49. The containment flaps 50, 52 can be constructed of a fibrous material which can be similar to the material forming the bodyside liner 28. Other conventional materials, such as polymer films, can also be employed.

The containment flaps 50, 52 can each include a base portion 64 and a projection portion 66. The base portion 64 can be bonded to the chassis 11, for example, to the bodyside liner 28 or the outer cover 26 as mentioned above. The base portion 64 can include a proximal end 64a and a distal end 64b. The projection portion 66 can be separated from the base portion 64 at the proximal end 64a of the base portion 64. As used in this context, the projection portion 66 is separated from the base portion 64 at the proximal end 64a of the base portion 64 in that the proximal end 64a of the base portion 64 defines a transition between the projection portion 66 and the base portion 64. The proximal end 64a of the base portion 64 can be located near the barrier adhesive 49. In some embodiments, the distal ends 64b of the base portion 64 can laterally extend to the respective longitudinal side edges 18, 20 of the absorbent article 10, 210. In other embodiments, the distal ends 64b of the base portion 64 can end laterally inward of the respective longitudinal side edges 18, 20 of the absorbent article 10, 210. The containment flaps 50, 52 can also each include a projection portion 66 that is configured to extend away from the body facing surface 19 of the chassis 11 at least in the crotch region 16 when the absorbent article 10, 210 is in a relaxed configuration, as illustrated in FIG. 5. The containment flaps 50, 52 can include a tack-down region 71 in either or both of the front waist region 12 and the rear waist region 14 where the projection portion 66 is coupled to the body facing surface 19 of the chassis 11.

It is contemplated that the containment flaps 50, 52 can be of various configurations and shapes, and can be constructed by various methods. For example, the containment flaps 50, 52 of FIG. 5 depict a vertical containment flap 50, 52 with a tack-down region 71 in both the front and rear waist regions 12, 14 where the projection portion 66 of each containment flap 50, 52 is tacked down to the bodyside liner 28 towards or away from the longitudinal axis 29 of the absorbent article 10, 210. However, the containment flaps 50, 52 can include a tack-down region 71 where the projection portion 66 of each of the containment flaps 50, 52 is folded back upon itself and coupled to itself and the bodyside liner 28 in a “C-shape” configuration, as is known in the art and described in U.S. Pat. No. 5,895,382 to Robert L. Popp et al. As yet another alternative, it is contemplated that the containment flaps 50, 52 could be constructed in a “T-shape” configuration, such as described in U.S. Pat. No. 9,259,362 by Robert L. Popp et al. Such a configuration can also include a tack-down region 71 in either or both of the front and rear waist regions 12, 14, respectively. Of course, other configurations of containment flaps 50, 52 can be used in the absorbent article 10, 210 and still remain within the scope of this disclosure.

The containment flaps 50, 52 can include one or more flap elastic members 68, such as the two flap elastic strands depicted in FIG. 5. Suitable elastomeric materials for the flap elastic members 68 can include sheets, strands or ribbons of natural rubber, synthetic rubber, or thermoplastic elastomeric materials. Of course, while two elastic members 68 are shown in each containment flap 50, 52, it is contemplated that the containment flaps 50, 52 can be configured with one or three or more elastic members 68. Alternatively or additionally, the containment flaps 50, 52 can be composed of a material exhibiting elastomeric properties itself.

The flap elastic members 68, as illustrated in FIG. 5, can have two strands of elastomeric material extending longitudinally in the projection portion 66 of the containment flaps 50, 52, in generally parallel, spaced relation with each other. The elastic members 68 can be within the containment flaps 50, 52 while in an elastically contractible condition such that contraction of the strands gathers and shortens the projection portions 66 of the containment flaps 50, 52 in the longitudinal direction 30. As a result, the elastic members 68 can bias the projection portions 66 of the containment flaps 50, 52 to extend away from the body facing surface 45 of the absorbent assembly 44 in a generally upright orientation of the containment flaps 50, 52, especially in the crotch region 16 of the absorbent article 10, 210, when the absorbent article 10 is in a relaxed configuration.

During manufacture of the containment flaps 50, 52 at least a portion of the elastic members 68 can be bonded to the containment flaps 50, 52 while the elastic members 68 are elongated. The percent elongation of the elastic members 68 can be, for example, 110% to 350%. In one embodiment, the elastic members 68 can be coated with adhesive while elongated to a specified length prior to attaching to the elastic members 68 to the containment flaps 50, 52. In a stretched condition, the length of the elastic members 68 which have adhesive coupled thereto can provide an active flap elastic region 70 in the containment flaps 50, 52, as labeled in FIG. 2, which will gather upon relaxation of the absorbent article 10. The active flap elastic region 70 of containment flaps 50, 52 can be of a longitudinal length that is less than the length of the absorbent article 10, 210. In this exemplary method of bonding the elastic members 68 to the containment flaps 50, 52, the portion of the elastic members 68 not coated with adhesive, will retract after the elastic members 68 and the absorbent article 10 are cut in manufacturing to form an individual absorbent article 10. As noted above, the relaxing of the elastic members 68 in the active flap elastic region 70 when the absorbent article 10, 210 is in a relaxed condition can cause each containment flap 50, 52 to gather and cause the projection portion 66 of each containment flap 50, 52 to extend away from the body facing surface 19 of the chassis 11 (e.g., the body facing surface 45 of the absorbent assembly 44 or the body facing surface 56 of the bodyside liner 28), as depicted in FIG. 5.

Of course, the elastic members 68 can be bonded to the containment flaps 50, 52 in various other ways as known by those of skill in the art to provide an active flap elastic region 70, which is within the scope of this disclosure. Additionally, the active flap elastic regions 70 can be shorter or longer than depicted herein, including extending to the front waist edge 22 and the rear waist edge 24, and still be within the scope of this disclosure.

### Leg Elastics:

Leg elastic members 60, 62 can be secured to the outer cover 26, such as by being bonded thereto by laminate adhesive, generally laterally inward of the longitudinal side edges, 18 and 20, of the absorbent article 10, 210. The leg elastic members 60, 62 can form elasticized leg cuffs that further help to contain body exudates. In an embodiment, the leg elastic members 60, 62 may be disposed between inner and outer layers (not shown) of the outer cover 26 or between other layers of the absorbent article 10, for example, between the base portion 64 of each containment flap 50, 52 and the bodyside liner 28 as depicted in FIG. 5, between the base portion 64 of each containment flap 50, 52 and the outer cover 26, or between the bodyside liner 28 and the outer cover 26. The leg elastic members 60, 62 can be one or more elastic components near each longitudinal side edge 18, 20. For example, the leg elastic members 60, 62 as illustrated herein each include two elastic strands. A wide variety of elastomeric materials may be used for the leg elastic members 60, 62.

Suitable elastomeric materials can include sheets, strands or ribbons of natural rubber, synthetic rubber, or thermoplastic elastomeric materials. The elastomeric materials can be stretched and secured to a substrate, secured to a gathered substrate, or secured to a substrate and then elasticized or shrunk, for example, with the application of heat, such that the elastic retractive forces are imparted to the substrate. Additionally, it is contemplated that the leg elastic members 60, 62 can be formed with the containment flaps 50, 52, and then attached to the chassis 11 in some embodiments. Of course, the leg elastic members 60, 62 can be omitted from the absorbent article 10, 210 without departing from the scope of this disclosure.

### Waist Containment Member:

In an embodiment, the absorbent article 10, 210 can have one or more waist containment members 54. The waist containment member(s) 54 can be disposed in the rear waist region 14 as illustrated in FIGS. 1-5. In general, the waist containment member 54 can help contain and/or absorb body exudates, especially low viscosity fecal matter, and as such, can be preferred to be in the rear waist region 14. In some embodiments, the absorbent article 10, 210 can have a waist containment member 54 disposed in the front waist region 12. A waist containment member 54 in the front waist region 12 can help contain and/or absorb body exudates, such as urine, in the front waist region 12. Although not as prevalent as in the rear waist region 14, in some circumstances, fecal material may also spread to the front waist region 12, and thus, a waist containment member 54 disposed in the front waist region 12 can help contain and/or absorb body exudates as well. In other embodiments, the absorbent article 10, 210 can have a waist containment member 54 in both the rear waist region 14 and the front waist region 12.

The waist containment member 54 can be disposed on the body facing surface 45 of the absorbent assembly 44. In some embodiments, such as in embodiments illustrated in FIGS. 1-2 and 5, the waist containment member 54 can be disposed on the body facing surface 56 of the bodyside liner 28. However, in some embodiments, such as the absorbent article 210 in FIG. 4, the waist containment member 54 can be disposed on a body facing surface 58 of the rear waist panel 15.

The waist containment member 54 can include a first longitudinal side edge 72 and a second longitudinal side edge 74. The first longitudinal side edge 72 can be opposite from the second longitudinal side edge 74. The distance between the first longitudinal side edge 72 and the second longitudinal side edge 74 can define a width 51 of the waist containment member 54 in the lateral direction 32, as shown in FIG. 2.

As illustrated in FIGS. 2 and 5, the waist containment member 54 can be configured such that the first longitudinal side edge 72 can be disposed laterally outward of the proximal end 64a of the base portion 64 of the containment flap 50. Similarly, the waist containment member 54 can be configured such that the second longitudinal side edge 74 can be disposed laterally outward of the proximal end 64a of the base portion 64 of the containment flap 52. The waist containment member 54 can be configured such that the width 51 of the waist containment member 54 can be greater than a lateral distance between longitudinal extending fold lines 25a, 25b, as shown in FIGS. 2 and 4.

The waist containment member 54 can also include a proximal portion (not shown) and a distal portion 78. The proximal portion can be coupled to the body facing surface 19 of chassis 11 (e.g., the body facing surface 45 of the absorbent assembly 44 or the body facing surface 56 of the bodyside liner 28) whereas the distal portion 78 of the waist containment member 54 can be free to move with respect to the chassis 11 and the absorbent assembly 44 when the absorbent article 10, 210 is in the relaxed configuration, such as shown in FIG. 5. When the waist containment member 54 is in a relaxed configuration, the distal portion 78 extends away from the chassis 11 and absorbent assembly 44 in a vertical direction, which is perpendicular to the plane defined by the longitudinal axis 29 and the lateral axis 31. A fold 79a can separate the proximal portion from the distal portion 78 of the waist containment member 54. As used in this context, the fold 79a separates the proximal portion from the distal portion 78 in that the fold 79a defines a transition between the proximal portion and the distal portion 78.

In some embodiments, the proximal portion of the waist containment member 54 can be coupled to the body facing surface 56 of the bodyside liner 28. In other embodiments, the proximal portion of the waist containment member 54 can be coupled to the body facing surface 58 of the rear waist panel 15. The proximal portion can be coupled to the body facing surface 45 by an adhesive, by pressure bonding, by ultrasonic bonding, by thermal bonding, and combinations thereof.

Because the distal portion 78 of the waist containment member 54 can freely move with respect to the absorbent assembly 44 when the absorbent article 10, 210 is in the relaxed configuration, the distal portion 78 can help provide a containment pocket 82 when the absorbent article 10, 210 is in the relaxed configuration. The containment pocket 82 can help provide a barrier to contain and/or can help absorb body exudates. The containment pocket 82 can be especially beneficial for containing and/or absorbing low viscosity fecal matter, which can be prevalent in younger children. The first longitudinal side edge 72 can be disposed laterally outward of the proximal end 64a of the base portion 64 of the containment flap 50, and thus, the containment pocket 82 can extend laterally outward of the proximal end 64a of the containment flap 50. Similarly, the second longitudinal side edge 74 can be disposed laterally outward of the proximal end 64a of the base portion 64 of the containment flap 52 and the containment pocket 82 can extend laterally outward of the proximal end 64a of the containment flap 52. Such a configuration provides waist containment member 54 with a wide containment pocket 82 to contain and/or absorb body exudates.

To help prevent lateral flow of body exudates that are contained by the containment pocket 82 of the waist containment member 54, the distal portion 78 of the waist containment member 54 can be bonded to the proximal portion of the waist containment member 54 and/or the body facing surface 19 of the chassis 11 near the first and second longitudinal side edges 72, 74, respectively. For example, FIG. 5 depicts tack-down regions 84 where the distal portion 78 of the waist containment member 54 can be bonded to the proximal portion of the waist containment member 54 and/or the body facing surface 19 of the chassis 11.

In preferred embodiments, the waist containment member 54 can include at least one elastic member and even more elastic members in further embodiments. Generally, the elastic member can span substantially from the first longitudinal side edge 72 to the second longitudinal side edge 74 of the waist containment member 54. The elastic member can be disposed in the distal portion 78 of the waist containment member 54, and preferably, is located near a free edge 88 of the distal portion 78 of the waist containment member 54.

A wide variety of elastomeric materials may be used for the elastic member(s) in the waist containment member 54. Suitable elastomeric materials can include sheets, strands or ribbons of natural rubber, synthetic rubber, elastic foams, or thermoplastic elastomeric materials (e.g., films). The elastomeric materials can be stretched and secured to a substrate forming the waist containment member 54, secured to a gathered substrate, or secured to a substrate and then elasticized or shrunk, for example, with the application of heat, such that the elastic retractive forces are imparted to the substrate forming the waist containment member 54.

The waist containment member 54 can be disposed to be coupled to the chassis 11 by being placed either over the containment flaps 50, 52 or under the containment flaps 50, 52. More specifically, the waist containment member 54 can be disposed on the body facing surface 19 of the chassis 11 such that the proximal portion of the waist containment member 54 is disposed over the base portion 64 of the first and the second containment flaps 50, 52, respectively. Alternatively, the waist containment member 54 can be disposed on the body facing surface 19 of the chassis 11 such that the proximal portion of the waist containment member 54 is disposed under the base portion 64 of the first and the second containment flaps 50, 52, respectively. Both configurations can provide advantages to the functioning of the waist containment member 54 to contain and/or absorb body exudates.

Where the proximal portion of the waist containment member 54 is disposed over the base portion 64 of the containment flaps 50, 52, the containment flaps 50, 52 can have an active flap elastic region 70 that longitudinally overlaps with the distal portion 78 of the waist containment member 54 when the absorbent article 10 is in the stretched, laid flat configuration, such as illustrated in FIG. 2. Additionally or alternatively, the tack-down region 71 may not extend from the rear waist edge 24 to the free edge 88 of the distal portion 78 of the waist containment member 54, such as illustrated in FIG. 2.

Where the proximal portion of the waist containment member 54 is disposed under the base portion 64 of the containment flaps 50, 52, the tack-down region 71 of the projection portion 66 of each of the containment flaps 50, 52 may longitudinally overlap with the distal portion 78 of the waist containment member 54. In some of these embodiments, the tack-down region 71 of projection portion 66 of each of the containment flaps 50, 52 can extend to the free edge 88 of the waist containment member 54 to further assist in containing exudates to the containment pocket 82 created by the waist containment member 54.

The waist containment member 54 can be comprised of a variety of materials. In a preferred embodiment, the waist containment member 54 can be comprised of a spunbond-meltblown-spunbond (“SMS”) material. However it is contemplated that the waist containment member 54 can be comprised of other materials including, but not limited to, a spunbond-film-spunbond (“SFS”), a bonded carded web (“BCW”), or any non-woven material. In some embodiments, the waist containment member 54 can be comprised of a laminate of more than one of these exemplary materials, or other materials. In some embodiments, the waist containment member 54 can be comprised of a liquid impermeable material. In some embodiments, the waist containment member 54 can be comprised of a material coated with a hydrophobic coating. The basis weight of the material forming the waist containment member 54 can vary, however, in a preferred embodiment, the basis weight can be between 8 gsm to 120 gsm, not including the elastic members 86 in the waist containment member 54. More preferably, the basis weight of the material comprising the waist containment member 54 can be between 10 gsm to 40 gsm, and even more preferably, between 15 gsm to 25 gsm.

### Fastening System:

In an embodiment, the absorbent article 10 can include a fastening system. The fastening system can include one or more back fasteners 91 and one or more front fasteners 92. The embodiments shown in FIGS. 1 and 2 depict embodiments with one front fastener 92. Portions of the fastening system may be included in the front waist region 12, rear waist region 14, or both.

The fastening system can be configured to secure the absorbent article 10 the waist of the wearer in a fastened condition as shown in FIG. 1 and help maintain the absorbent article 10 in place during use. In an embodiment, the back fasteners 91 can include one or more materials bonded together to form a composite ear as is known in the art. For example, the composite fastener may be composed of a stretch component 94, a nonwoven carrier or hook base 96, and a fastening component 98, as labeled in FIG. 2. As shown in FIG. 5, in some embodiments the waist containment member 54 can extend to back fasteners 91. In some embodiments, the waist containment member 54 can be coupled to the stretch component 94 of the back fasteners 91, either directly or indirectly. In some embodiments, the waist containment member 54 can extend to the longitudinal side edges 18, 20 of the absorbent article 10, 210.

### Absorbent Core:

The absorbent core 34 can be suitably constructed to be generally compressible, conformable, pliable, non-irritating to the wearer's skin and capable of absorbing and retaining liquid body exudates. The absorbent core 34 can be manufactured in a wide variety of sizes and shapes (for example, rectangular, trapezoidal, T-shape, I-shape, hourglass shape, etc.) and from a wide variety of materials. The size and the absorbent capacity of the absorbent core 34 should be compatible with the size of the intended wearer (infants to adults) and the liquid loading imparted by the intended use of the absorbent article 10, 210. The absorbent core 34 can have a length and width that can be less than or equal to the length and width of the absorbent article 10, 210.

The absorbent core 34 is generally composed of absorbent material, such as fibrous absorbent material and/or, superabsorbent material, and can include further materials such as binder materials, surfactants, selected hydrophobic and hydrophilic materials, pigments, lotions, odor control agents or the like, as well as combinations thereof. According to some examples, the absorbent core 34 is a matrix of cellulosic fluff and superabsorbent material. In other examples, the absorbent material of the absorbent core 34 comprises only superabsorbent material. The absorbent core 34 may be constructed of a single layer of materials, or in the alternative, may be constructed of two or more layers of materials.

When composed at least partially of fibrous material, various types of wettable, hydrophilic fibers can be used in the absorbent core 34. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber.

When composed at least partially of superabsorbent materials, such superabsorbent materials can be selected from natural, synthetic, and modified natural polymers and materials. The superabsorbent materials can be inorganic materials, such as silica gels, or organic compounds, such as cross-linked polymers.

If a spacer layer 48 is present, the absorbent core 34 can be disposed on the spacer layer 48 and superposed over the outer cover 26. The spacer layer 48 can be bonded to the outer cover 26, for example, by adhesive. In some embodiments, a spacer layer 48 may not be present and the absorbent core 34 can directly contact the outer cover 26 and can be directly bonded to the outer cover 26. However, it is to be understood that the absorbent core 34 may be in contact with, and not bonded with, the outer cover 26 and remain within the scope of this disclosure. In an embodiment, the outer cover 26 can be composed of a single layer and the absorbent core 34 can be in contact with the singer layer of the outer cover 26. In some embodiments, at least a portion of a layer, such as but not limited to, a fluid transfer layer 46 and/or a spacer layer 48, can be positioned between the absorbent core 34 and the outer cover 26, such as illustrated in FIG. 5. The absorbent core 34 can be bonded to the fluid transfer layer 46 and/or the spacer layer 48.

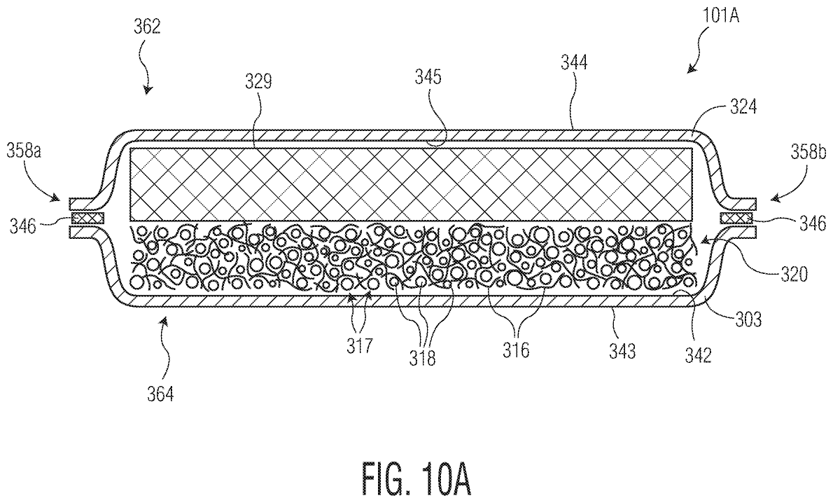

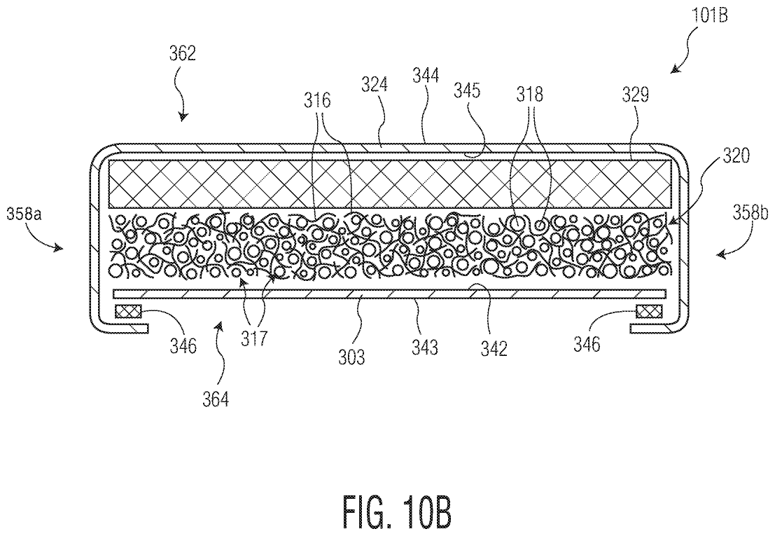

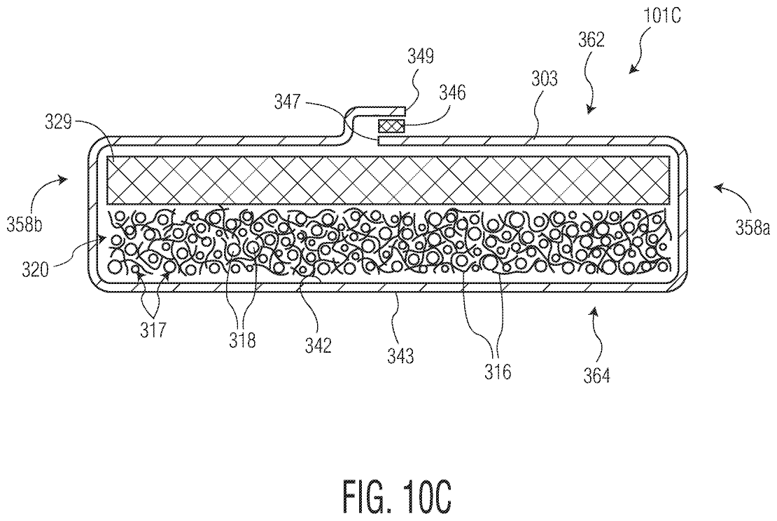

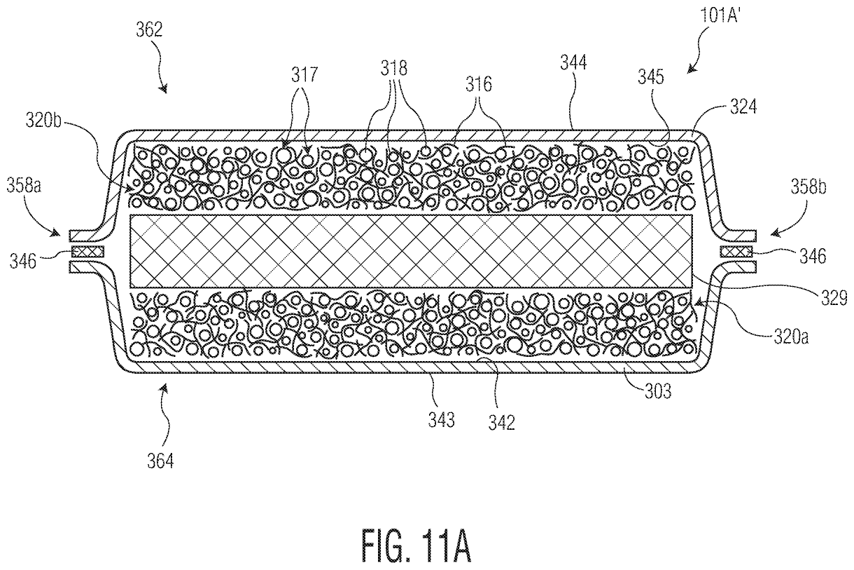

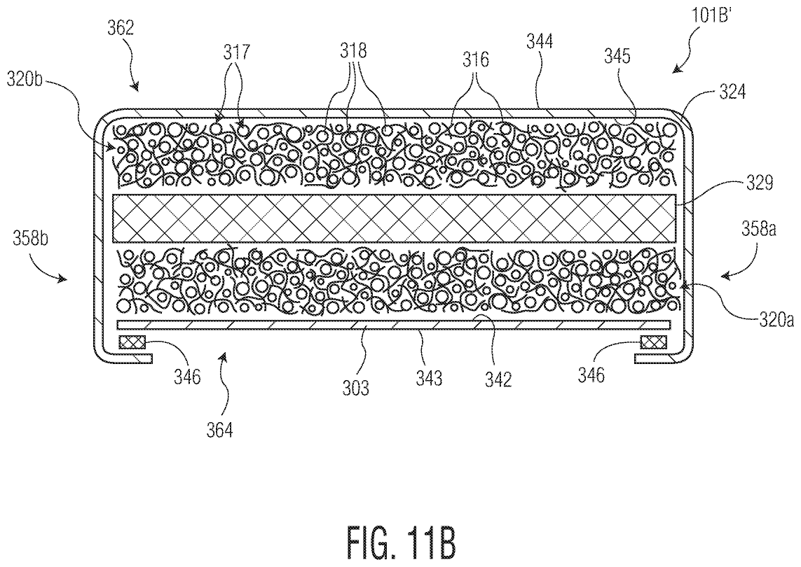

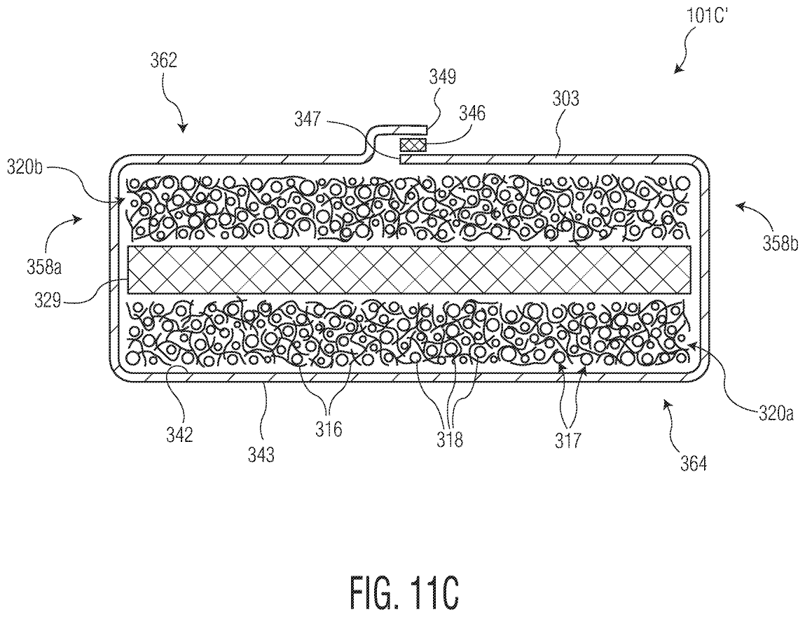

According to some aspects of the present disclosure, the absorbent core 34, or at least one component of the absorbent core 34, may comprise an absorbent structure, such as those described in more detail with respect to FIGS. 10A-10C and 11A-11C. In some examples, the absorbent structure is the entirety of the absorbent core 34. Alternative examples include where the absorbent structure comprises only a portion of the absorbent core 34. In such examples, the absorbent structure may be contained within the absorbent core 34 along with other material, such as one or more web materials and/or additional absorbent materials. Such other materials, along with absorbent structure, which in total form the absorbent core 34, may generally be identified as being part of the absorbent core 34 by their inclusion under fluid transfer layer 46—which may or may not wrap around side edges of the absorbent core 34 in different embodiments—and above the outer cover 26 or spacer layer 48. By contrast, the absorbent core 34 and the fluid transfer layer 46, disposed between the spacer layer 48 or the outer cover 26 and the bodyside liner 28, may together comprise the absorbent system of the articles 10, 210.

In particular examples, such as those of FIGS. 10A-10C and 11A-11C, the absorbent material content of the absorbent structure can comprise mostly superabsorbent material, by weight of the absorbent material of the absorbent structure. For example, the absorbent material content of the absorbent structure, by weight of the absorbent material of the absorbent structure, can comprise greater than 80% superabsorbent material, greater than 85% superabsorbent material, greater than 90% superabsorbent material, greater than 95% superabsorbent material. In such embodiments, the remaining absorbent material content may comprise fibrous absorbent material, such as cellulosic fibers, or any other suitable absorbent material. Additional embodiments of the structures of FIGS. 10A-10C and 11A-11C where the absorbent material content of the absorbent structure, by weight of the absorbent material of the absorbent structure, comprises 100% superabsorbent material, providing a for cellulose-free absorbent structure.

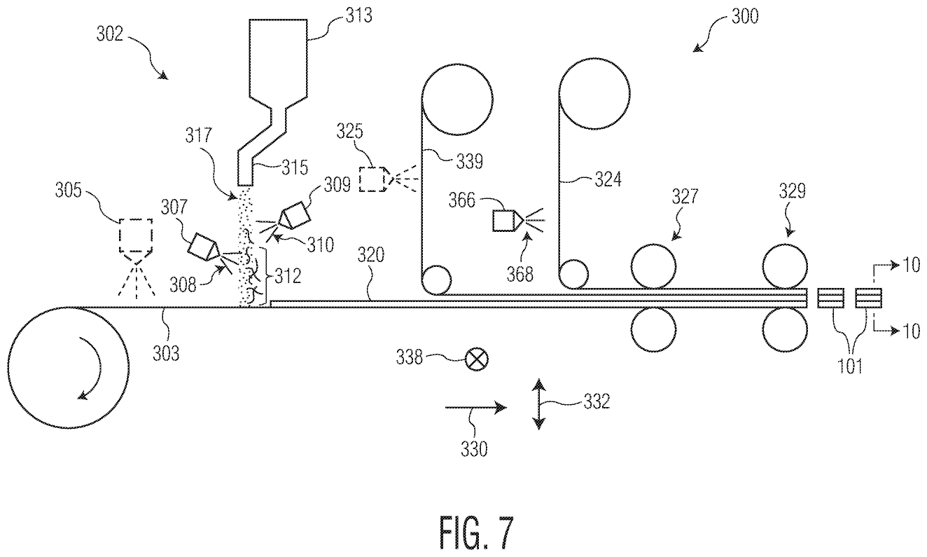

Absorbent structures according to the present disclosure may be formed according to the processes disclosed herein, such as processes 300, 400 detailed in FIGS. 7-9. Such absorbent structures formed by processes 300, 400 may advantageously provide greater thinness, flexibility, superabsorbent material capture, intake and/or rewet performance, and less overall material usage than absorbent structures formed by different processes and/or comprising different material or different relative amounts of material. Although the FIGS. 1-5 focus on description of a diaper absorbent article 10, 210, it should be understood that the absorbent structures of the present disclosure may be used in any absorbent article-including but not limited to diapers, diaper pants, training pants, youth pants, swim pants, feminine hygiene products, including, but not limited to, menstrual pads or pants, incontinence products and other adult care garments, medical garments, surgical pads and bandages, other personal care or health care garments, and the like.

FIG. 7 is an exemplary schematic depiction of absorbent structure formation process 300 for forming absorbent structures such as structures 101. Process 300 includes unwinding web material 303 and moving the web material 303 in a machine direction 330. In some particular examples, the web material 303 is a corewrap material of an absorbent structure. The web material 303 moves int eh machine direction 330 and arrives at an absorbent material deposition station 302. At the absorbent material deposition station 302, superabsorbent material 317, for example superabsorbent particles, intermixes with one or more adhesives 308, 310 prior to depositing onto the web material 303, for example in a mixing region 312, and ultimately deposits onto the web material 303.

The superabsorbent material 317 flows from hopper 313 and through chute 315 toward the web material 303. The hopper 313 may be a bulk solid pump or feeder configured to maintain a consistent flow of the superabsorbent material 317 through the absorbent material deposition station 302. The flow rate of the superabsorbent material 317 out of the hopper 313 may be adjustable such that the hopper 313 can deliver different amounts of superabsorbent material 317, resulting in different basis weights of superabsorbent material 317 in the finished absorbent structures 101. Such differences in basis weights of superabsorbent material 317 may allow the formed absorbent structures 101 to be used in different absorbent end uses-such as in diapers, feminine articles, adult care garments, bandages and the like.

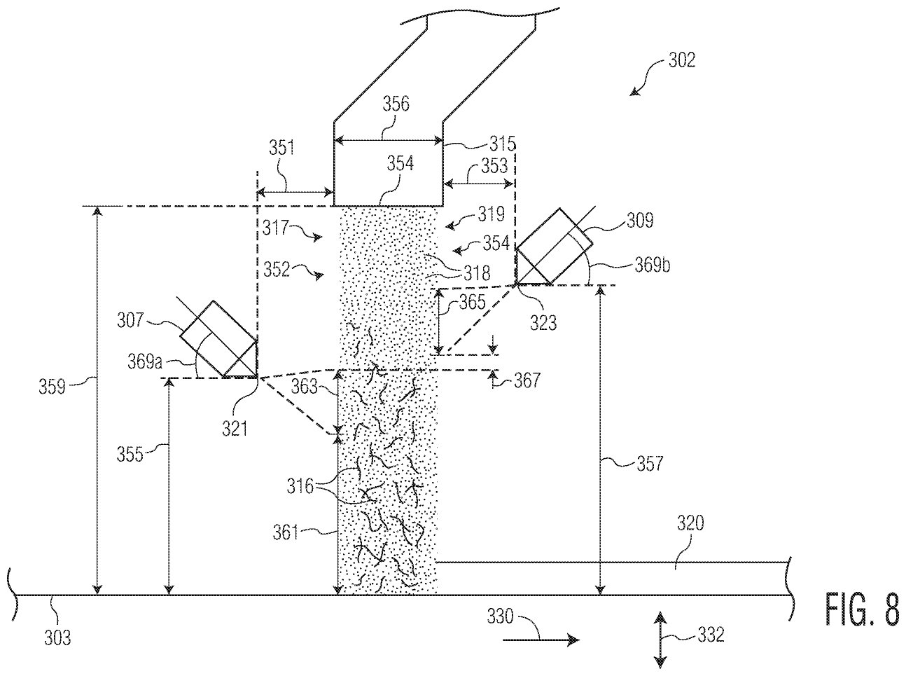

The chute 315 has a chute end 354 (as seen in FIG. 8) that is shown oriented in the vertical direction 332 such that the superabsorbent material 317, shown as individual particles 318 in FIG. 8, exits the chute 315 falling substantially in the vertical direction 332. The superabsorbent material 317 may preferably be fed through the absorbent material deposition station 302 by gravity, without any pneumatic force. As used herein, the vertical direction 332 is used to denote a direction perpendicular to the web material 303. The machine direction 330 is a direction parallel within the web material 303 and, accordingly, is perpendicular to the vertical direction 332. In examples where the web material 303 is oriented in a horizontal direction with respect to gravity (e.g. perpendicular to the direction of gravity), the vertical direction 332 may be substantially aligned with respect to gravity. However, in other examples, the vertical direction 332 may be at an angle with respect to gravity—for example angles of up to 25 degrees difference with respect to gravity may be suitable for the vertical direction 332. Accordingly, in such embodiments, the web material 303 may not be moving in a horizontal direction with respect to gravity and the superabsorbent material 317 would fall toward the web material 303 having a direction including a component in both the vertical direction 332 and the machine direction 330 (or potentially opposite the machine direction 330).

Additionally, regardless of the orientation of the vertical direction 332 with respect to gravity, the chute 315 may further be oriented in a non-perpendicular fashion with respect to the web material 303. For instance, the chute end 354 may be oriented perpendicular with respect to the web material 303 (as shown in FIG. 8) or it may be oriented so as to form an angle of greater than 0 degrees and less than 25 degrees with respect to a direction perpendicular to the web material 303.

In general, the amount of superabsorbent material 317 fed through the absorbent material deposition station 302 can be configured to result in absorbent structures 101 comprising superabsorbent material 317 disposed in amounts between 50 gsm and 1000 gsm, or between 100 gsm and 1000 gsm, or between 150 gsm and 1000 gsm, or between 200 gsm and 800 gsm, or between 250 gsm and 800 gsm, or between 300 gsm and 700 gsm, or between 350 gsm and 700 gsm, or between 400 gsm and 700 gsm, or between 450 gsm and 700 gsm, or between 500 gsm and 700 gsm, or between 400 gsm and 600 gsm, or between 500 gsm and 600 gsm. Such superabsorbent material 317 basis weight values for absorbent structures may be particularly suitable for use in absorbent garments and feminine hygiene products. Although, further absorbent structures that may be formed according to aspects of the present disclosure can have even smaller basis weights of superabsorbent material 317, such as between 5 gsm and 50 gsm, or 5 gsm and 30 gsm, or between 10 gsm and 30 gsm.

The chute opening 354 has an opening width 356 (as measured where the superabsorbent material 317 exits the chute 315) in the machine direction 330. The opening width 356 may be between 2 mm and 30 mm, or between 5 mm and 25 mm, or between 5 mm and 20 mm, or between 7 mm and 15 mm. More specifically, opening widths 356 of between 2 mm and 10 mm are preferred when the amount of superabsorbent material 317 deposited by the absorbent material deposition station 302 is between 50 gsm and 300 gsm. Conversely, opening widths 356 of between 10 mm and 14 mm are preferred when the amount of superabsorbent material 317 deposited by the absorbent material deposition station 302 is between 300 gsm and 500 gsm, and opening widths 356 of between 14 mm and 20 mm are preferred when the amount of superabsorbent material 317 deposited by the absorbent material deposition station 302 is between 500 gsm and 1000 gsm.

These combinations of features—the gravity feed method and the chute opening width 356—may help to generate a “sheet” or “stream” of superabsorbent material 317 flowing toward the web material 303. The specified widths 356 may help to ensure that the stream 319 of superabsorbent material 317 has a sufficient width and/or density-particularly at the points where the adhesives 308 and/or 310 contact the stream 319, which can allow for the adhesives 308 and/or 310 to better penetrate the stream 319 and intermix with the superabsorbent material 317. These configurations can help to drive beneficial properties of the resulting absorbent structures 101, as described in more detail below. In some further embodiments, air streams or air curtains may be used to help shape the stream 319 and/or to maintain a desired width and/or density of the stream. In such embodiments, the superabsorbent material 317 might be directed toward the web material 303 to some extent faster than solely by gravity, but such embodiments would still be considered to comprise a gravity feed system because the superabsorbent material 317 is not pneumatically or otherwise forced from the chute end 354.

As the superabsorbent material 317 falls toward the web material 303, adhesive applicators 307 and/or 309 spray adhesive 308 and/or 310 toward the falling superabsorbent material 317. The adhesive 308 and/or 310 intermixes with the falling superabsorbent material 317 prior to the mixture of the superabsorbent material 317 and the adhesive 308 and/or 310 depositing onto the web material 303. FIG. 8 is a close-up schematic depiction of the absorbent material deposition station 302, showing more details regarding the adhesive applicators 307 and/or 309, the adhesives 308 and/or 310.

The adhesives 308 and/or 310 applied by adhesive applicators 307 and/or 309 may generally be applied at add-on percentages of less than 7%, or less than 6%, or less than 5%, or less than 4%, or less than 3%, or less than 2%. In other embodiments, the add-on percentages may be between 1% and 7%, or between 1.5% and 7%, or between 2% and 6%, or between 2% and 5.5%, or between 2% and 5%, or between 2% and 4.5%, or between 2% and 4%, or between 2.5% and 4%, or between 3% and 4%. As used herein, the term “add-on” amount or percentage is the amount of adhesive added such that a resulting weight of the adhesive within the absorbent structure has the desired relation to a weight of the absorbent material within such absorbent structure. As one illustrative example, where the superabsorbent material 317 is disposed in an absorbent structure at a basis weight of 500 gsm, and where the adhesives 308 and/or 310 were applied at a combined add-on rate of 5%, the resulting basis weight of the adhesive 308 and/or 310 in the formed absorbent structure would be 25 gsm (5% of the 500 gsm basis weight of the superabsorbent material 317).

As shown in FIGS. 7-9, the absorbent material deposition station 302 can comprise two adhesive applicators 307 and 309. The first adhesive applicator 307 may be positioned upstream (relative to the process direction 330) of the chute 315 while the second adhesive applicator 309 may be positioned downstream of the chute 315. The superabsorbent material 317 may form a stream 319 of superabsorbent material 317 as it falls toward the web material 303. Where the adhesive applicator 307 is positioned on the upstream side of the chute 315, the adhesive applicator 307 is configured to spray the first adhesive 308 at the first side 352 of the stream 319 of superabsorbent material 317.

The adhesive applicator 307 may be configured to spray the first adhesive 308 such that the first adhesive 308 contacts the first side 352 of the stream 319 of superabsorbent material 317 along a portion of the stream 319 having a length 363 along the stream 319. In some embodiments, the length 363 may be insubstantial in that the first adhesive 308 may be sprayed as a stream which has minimal-to-no spread. However, in other embodiments, the first adhesive 308 may have some spread and accordingly the length 363 could be between 2 mm and 10 mm, or between 2 mm and 6 mm, or between 2 mm and 4 mm.

In order to allow sufficient time for the first adhesive 308 to intermix with the stream 319 of the superabsorbent material 317 prior to the mixture of the first adhesive 308 and the superabsorbent material 317 depositing onto the web material 303, the first adhesive 308 desirably contacts the stream 319 at a first contact point located a distance 361 away from the web material 303. The distance 361 may be between 4 mm and 40 mm, or between 4 mm and 35 mm, or between 5 mm and 30 mm, or between 6 mm and 25 mm. Where the first adhesive 308 is sprayed in a spread fashion and contacts the stream 319 along some length 363, the first contact point, and accordingly the distance 361, is measured with respect to the center of the length 363 along which the first adhesive 308 contacts the stream 319.

To achieve such distances 361, the nozzle 321 is positioned a distance 355 away from the web material 303 and a distance 351 away from the chute 315. These distances 355, 351 may be adjusted to achieve the desired distance 361. As some non-limiting examples, the distance 355 may generally be between 5 mm and 40 mm, or between 10 mm and 30 mm. As a comparison, the chute 315 may be positioned a distance 359 away from the web material 303. The distance 359 may be between 50 mm and 90 mm, or between 60 mm and 80, or between 70 mm and 80 mm. Distances 359 higher than 70 mm, or 80 mm, or 90 mm may result in undesirable spreading of the stream 319. Distances lower than 60 mm or 50 mm may result in there being insufficient space between the chute 315 and the web material 303 to allow for sufficient mixing of the superabsorbent material 317 and the first adhesive 308 (or the second adhesive 310 described in more detail below).

It has been further found that the angle 369a at which the nozzle 321 is oriented with respect to the machine direction 330 may be important in achieving a desired level of mixing between the first adhesive 308 and the stream 319. Preferably, the angle 369a is between 40 degrees and 80 degrees, or between 45 degrees and 75 degrees, or between 50 degrees and 70 degrees.

The adhesive applicator 309 may be configured similarly to the adhesive applicator 307. The adhesive applicator 309 can spray the second adhesive 310 such that the second adhesive 310 contacts the second side 354 of the stream 319 of superabsorbent material 317 along a portion of the stream 319 having a length 365 along the stream 319. Accordingly, the length 365 may be insubstantial in that the second adhesive 310 may be sprayed as a stream which has minimal-to-no spread. In other configurations, the second adhesive 310 may have some spread such that the length 365 may vary between 2 mm and 10 mm, or between 2 mm and 6 mm, or between 2 mm and 4 mm.

To allow sufficient time for the second adhesive 310 to intermix with the stream 319 of the superabsorbent material 317 prior to the mixture of the second adhesive 310 and the superabsorbent material 317 depositing onto the web material 303, the second adhesive 310 preferably contacts the stream 319 at a second contact point on the stream 319 located a distance away from the web material 303 equal to distance 367 added to distance 361. The distance 367 plus distance 361 may generally be between 4 mm and 40 mm, or between 4 mm and 35 mm, or between 5 mm and 30 mm, or between 6 mm and 25 mm. Additionally, where the second adhesive 310 is sprayed in a spread fashion and contacts the stream 319 along some length 365, the second contact point, as well as the distance 367 added to the distance 361, is measured with respect to the center of the length 365 along which the second adhesive 310 contacts the stream 319 (and with respect to the center of the length 363 if the first adhesive 308 contact the stream 319 for some appreciable length 363).

It can be understood that the distance 361 and the distance 367 added to the distance 361 overlap in their preferred ranges. According to some preferred embodiments, the distance 361 is less than the distance 367 added to the distance 361. For example, it may be preferred that the applicator 307 is positioned closer to the web material 303 than the applicator 309. In such embodiments, the distance 361 may be preferred to be between 4 mm and 22 mm, or between 4 mm and 20 mm, or between 6 mm and 15 mm. The distance 367 added to the distance 361 may be greater than the distance 361 by between 5 mm and 15 mm, or between 6 mm and 13 mm, or between 6 mm and 11 mm, e.g. the distance 367 may be between 5 mm and 15 mm, or between 6 mm and 13 mm, or between 6 mm and 11 mm. In such embodiments, the distance 367 may represent a spacing between the first contact point where the first adhesive 308 contacts the stream 319 and the second contact point where the second adhesive 310 contacts the stream 319.

It has been found that spraying the adhesives 308 and/or 310 at the stream 319 may cause the stream 319 to bend in the direction of the spray. Without being limited by theory, it is thought that the force of the adhesives 308 and/or 310 contacting the stream and/or the optional pattern air supplied by the applicators 307 and/or 309 can cause this bending of the stream 319. Accordingly, where the first contact point where the first adhesive 308 contacts the stream 319 is at a lower point than the second contact point where the second adhesive 310 contacts the stream 319, the stream 319 may bend in the machine 330 just prior to depositing onto the web material 303. This bending of the stream 309 in the machine direction 330 helps to ensure a smooth deposition of mixture of the superabsorbent material 317 and the first adhesive 308 (and optionally the second adhesive 310), resulting in a more uniform mixture 320, which has many benefits in terms of capture and stabilization of the superabsorbent material 317, integrity of the resulting absorbent structure 101, and uniformity of the distribution of the superabsorbent material 317 and the first adhesive 308 (and optionally the second adhesive 310).

As with the nozzle 321, the nozzle 323 may be positioned a distance 357 away from the web material 303 and a distance 353 away from the chute 315 in order to achieve the desired distance 367 added to the distance 361. The angle 369b at which the nozzle 323 is oriented with respect to the web of material 303 may further be similar to the angle 369a. For example, the angle 369b may vary between 40 degrees and 80 degrees, or between 45 degrees and 75 degrees, or between 50 degrees and 70 degrees. In at least some embodiments, the angle 369a and the angle 369b can be the same, while in other embodiments the angles 369a, 369b are different.

The applicators 307 and/or 309 may be preferably configured to spray the adhesives 308 and/or 310 in a substantially random pattern. It has been found that spray patterns that are more randomized, irregular, or erratic may produce better results in terms of performance of the absorbent structures 101—such as in terms of capture and stabilization of the superabsorbent material 317, integrity of the resulting absorbent structure 101, and uniformity of the distribution of the superabsorbent material 317 and the adhesives 308 and/or 310. One such exemplary spray pattern is the pattern produced by the Universal™ Signature™ Spray Nozzles available from the Nordson Corporation having headquarters at 28601 Clemens Road, Westlake, OH 44145 USA. However, in other embodiments, different adhesive spray patterns which are more regular and less randomized, but still considered a random pattern, may be sufficient to produce absorbent structures 101 having desirable performance properties. It is further contemplated that some non-random spray patterns may also be sufficient to produce absorbent structures 101 having desirable performance properties.

Although shown in FIG. 8 as comprising two adhesive applicators 307 and/or 309, in some embodiments the absorbent material deposition station 302 may only comprise one of the adhesive applicators 307 and/or 309. Further, although shown, and described above, with the adhesive applicator 307 directing adhesive 308 to the first side 352 of the stream 319 (which is the upstream side of the stream 319) positioned closer to the web material 303 than the adhesive applicator 309, this orientation is not required in all embodiments. For example, in further embodiments, the adhesive applicator 307 may be positioned further away from the web material 303 than the adhesive applicator 309, while still being positioned on the upstream side of the stream 319. In any of these such embodiments, the distances between the first contact point and the second contact point with respect to each other and with respect to the web material described previously may be reversed. That is, the distance 361 may describe the distance between the second contact point and the web material 303 while the distance 367 added to the distance 361 may describe the distance between the first contact point and the web material 303 (with the distance 367 describing the distance between the first contact point and the second contact point).

As the web 303 passes the absorbent material deposition station 302, a deposited mixture 320 of the mixture of the adhesives 308 and/or 310 and the superabsorbent material 317 forms as a matrix of adhesive and superabsorbent material 317. Generally, the adhesives 308 and/or 310, within the mixing in the mixing region 312, form a mesh network with the superabsorbent material 317 captured therein. When this mesh network is deposited onto the web 303, the adhesives 308 and/or 310 forms a three-dimensional mesh network with the superabsorbent material 317 immobilized therein.

During the deposition of the mixture 320, vacuum energy may optionally be applied to the web material 303. For example, web material 303 may be supported by a forming surface-such as a forming belt or forming drum as is typical in the art. Vacuum energy may be applied to the forming surface such that air is drawn through the forming surface from the side where the web material 303 is located. Accordingly, the web material 303, along with the mixture 320 as it is falling toward the web material 303, is drawn to the forming surface due to the applied vacuum energy. Such vacuum energy may help to control the spread of the mixture 320 as it is falling toward the web material 303, thereby helping to form a relatively more uniform absorbent structure 101. It has been found that particularly high pressure differentials are preferred at the forming surface-above and beyond typical pressure differentials in the art. For example, it may be preferred that the vacuum energy produces a pressure differential of greater than 0.25 m of water at the forming surface. In further embodiments, it may be more preferable for even higher pressure differentials, such as greater than 0.35 m of water, or greater than 0.5 m of water, or greater than 0.65 m of water, as measured at the forming surface.

An internal reinforcing web material 329 may further be applied to the deposited mixture 320. The reinforcing web material 329 may generally be a lofty non-woven material and can provide structure to the absorbent structure 101. In the embodiment of process 300, only a single absorbent layer may be formed as part of the absorbent structure 101. In these embodiments, a further web material 324 may be applied to the reinforcing web material 329. An additional laminating adhesive 368 may be applied to the web material 324 (or the reinforcing web material 329 in other embodiments) by adhesive applicator 366 prior to combining the reinforcing web material 329 and the web material 324. Although, it should be understood that the adhesive applicator 368 is only optional and may not be present in some embodiments. Where present, the applied adhesive 368 may operate to more closely couple the web material 324 to the reinforcing web material 329.

According to some aspects of the present disclosure, the combination of the web material 303, the deposited mixture 320, the reinforcing web material 329, and the web material 324 may pass through one or more nip stations 327 to help compress the components together. In general, the nip station 327 may apply a pressure to the combination of the web material 303, the deposited mixture 320, the reinforcing web material 329, and the web material 324 of between 0.5 pounds per linear inch (PLI) (88 N/m) and 1.5 PLI (263 N/m), or between 0.75 PLI (131 N/m) and 1.25 PLI (219 N/m). Such pressures help to further connect the deposited mixture to the web materials 303, 324. Although not required in all embodiments, it may be preferred for the nip station 327 to be positioned in relatively close proximity to the material deposition station 302 such that the adhesives 308 and/or 310 is still open when the combination of the web material 303, the deposited mixture 320, and the web material 324 passes through the nip station 327.

After the one or more nip stations 327, the combination of the web material 303, the deposited mixture 320, the reinforcing web material 329, and the web material 324 may pass to a cutting station 329 where the connected length of the web material 303, the deposited mixture 320, the reinforcing web material 329, and the web material 324 is cut into individual absorbent structures 101. These individual absorbent structures 101 may then be combined into a manufacturing process for producing the various absorbent products described herein.

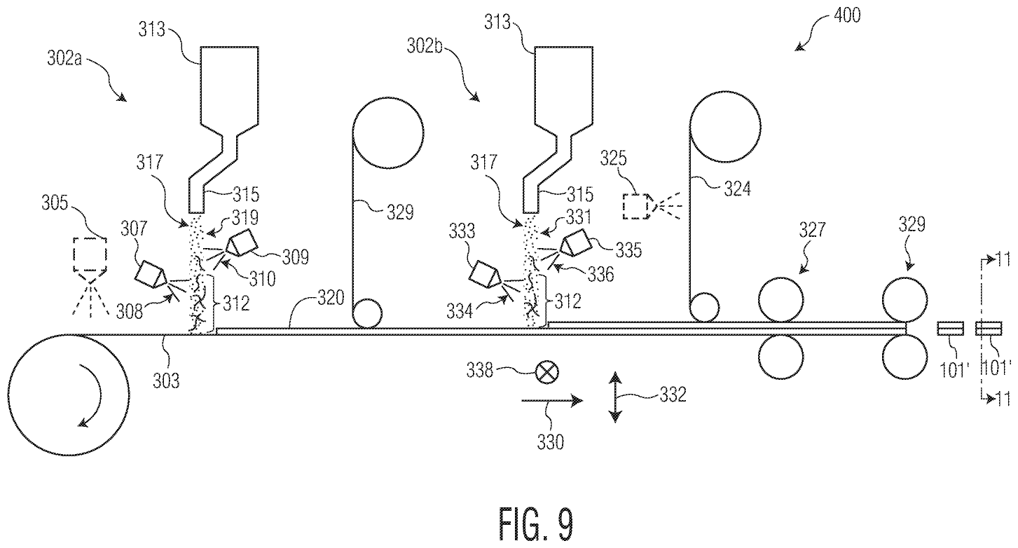

FIG. 9 depicts is an exemplary schematic depiction of an alternative absorbent structure formation process 400. The process 400 is similar to process 300, except the process 400 employs two absorbent material deposition stations 302a, 302b. It has been found that there are some advantages of employing two absorbent material deposition stations 302a, 302b over a single absorbent material deposition station 302, thus forming two separate absorbent layers. For example, as the desired amount of deposited superabsorbent material 317 gets higher, the lower the ability of a single absorbent material deposition station 302 to form an absorbent structure 101 having desired performance properties. For example, if the desired amount of deposited superabsorbent material 317 gets too high, a single absorbent material deposition station 302 may not be able to form a mixture of superabsorbent material 317 and adhesive which sufficiently immobilizes the superabsorbent material 317—particularly at desired low adhesive add-on amounts. For instance, in such examples, a superabsorbent material capture property of such formed absorbent structures 101 may fall below a desired value.

Conversely, the same desired amount of deposited superabsorbent material 317 may be able to be sufficiently immobilized by employing two absorbent material deposition stations 302a, 302b such that the resulting absorbent structures 101 have a desired superabsorbent material capture value. In such embodiments, the desired total amount of superabsorbent material 317 within the absorbent structure 101 may be split between the two absorbent material deposition stations 302a, 302b. Additionally, employing two absorbent material deposition stations 302a, 302b may allow for increased production rates-even at lower superabsorbent material 317 amounts and higher adhesive add-on amounts.

In the process 400, after the mixture of superabsorbent material 317 and adhesives 308 and/or 310 is deposited onto the web material 303 at absorbent material deposition station 302a (which may be equivalent to absorbent material deposition station 302 of FIGS. 7 and 8), the web material 303 and the deposited mixture of superabsorbent material 317 and adhesives 308 and/or 310 is laminated with the reinforcing web material 329.

After lamination with the reinforcing web material 329, the combination of the web material 303, the deposited mixture of superabsorbent material 317, adhesives 308 and/or 310, and the reinforcing web material 329 moves onto absorbent material deposition station 302b. Similar to absorbent material deposition station 302a, absorbent material deposition station 302b may be configured to direct a second stream 331 of superabsorbent material 317 toward the reinforcing web material 329. The absorbent material deposition station 302b may comprise adhesive applicators 333 and/or 335 which may spray adhesive 334 and/or 336 toward the second stream 331 of falling superabsorbent material 317. The adhesive 334 and/or 336 intermixes with the falling superabsorbent material 317 prior to the mixture of the superabsorbent material 317 of the second stream 331 and the adhesive 334 and/or 336 depositing onto the reinforcing web material 329. As with the deposited mixture 320, the adhesive 334 and/or 336 and superabsorbent material 317 at station 302b intermix prior to deposition onto the reinforcing web material 329 to form a mesh network with the superabsorbent material 317 captured therein. When this mesh network is deposited onto the reinforcing web material 329, the adhesives 334 and/or 336 form a three-dimensional mesh network with the superabsorbent material 317 immobilized therein. Finally, the web material 324 may be coupled to the deposited mixture of the adhesives 334 and/or 336 and the superabsorbent material 317.

According to some aspects of the present disclosure, the absorbent material deposition station 302b can comprise two adhesive applicators 333 and 335. With respect to the absorbent material deposition station 302b, the first adhesive applicator 333 (which may be the third adhesive applicator of the process 400) may be positioned upstream (relative to the process direction 330) of the chute 315 of the second deposition station 302b while the second adhesive applicator 335 (which may be the fourth adhesive applicator of the process 400) may be positioned downstream of the chute 315 of the second deposition station 302b. The adhesive applicator 333 is configured to spray the first adhesive 334 (which may be the third adhesive of the process 400) at a first side of the second stream 331 of superabsorbent material 317. The adhesive applicator 335 is configured to spray the second adhesive 336 (which may be the fourth adhesive of the process 400) at a second side of the second stream 331 of superabsorbent material 317.

Generally, the positions, locations, distances, and other features and optional components or features of the absorbent material deposition station 302 described with respect to FIG. 8 may be the same as for absorbent material deposition station 302a. Likewise, the absorbent material deposition station 302b may be the same or substantially similar to the absorbent material deposition station 302a. The absorbent material deposition station 302b may be positioned between 0.25 m and 3.0 m, or more preferably between 0.25 m and 2.0 m, or even more preferably between 0.25 m and 1.0 m.

Turning back to web materials 303 and 324, as shown in FIGS. 7 and 9, as shown, the web material 324 may be coupled to the reinforcing web material 329 or the deposited mixture of the superabsorbent material 317 and the adhesives 334, and/or 336 to form the absorbent structures 101. Some alternative embodiments according to aspects of the present disclosure may forgo the web material 324 altogether. In such embodiments, the web material 303 may be wide enough that, after the mixture 320 is deposited onto the web material 303, the web material 303 is wrapped around the mixture 320 to form the absorbent structure 101.

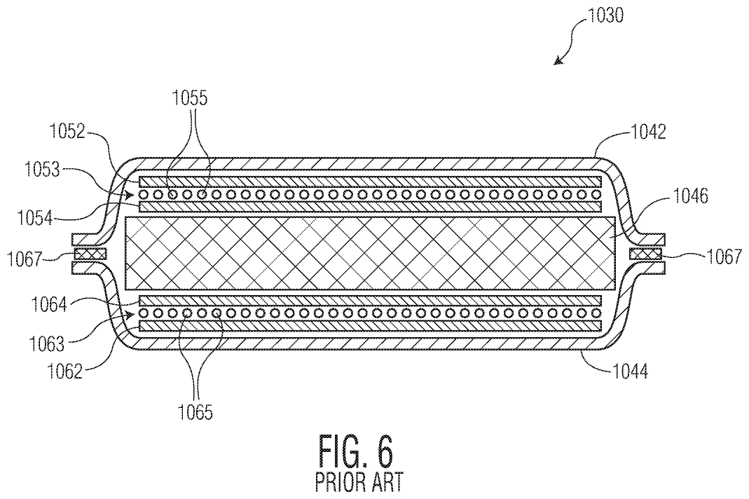

Notably, one feature absent from processes 300 and 400 is the lack of adhesive applied directly to the webs 303 and/or 324 in a region where the mixtures 320, 320a, 320b are deposited or contact the webs 303, 324, and/or the reinforcing web material 329. In at least some embodiments, the superabsorbent material 317 is able to be stabilized within the three-dimensional mesh network of the adhesives 308, 310, 334 and/or 336 to a sufficient degree that adhesives disposed between the matrices of superabsorbent material 317 and adhesives 308, 310, 334 and/or 336 comprising the first and second absorbent layers are not needed. Such embodiments generally allow for faster intake times relative to more traditional absorbent structures, such as structures 1030 of FIG. 6. For example, less adhesive overall may be utilized in the absorbent structures of the present disclosure. Adhesives are generally hydrophobic and block fluid flow into absorbent structures. Accordingly, a lesser amount of adhesive can help to improve intake times. Additionally, as will be described in more detail, it has been found that forming absorbent layers according to the processes 300, 400 can ensure a lesser amount of superabsorbent material 317 penetrates into the reinforcing web material 329 as compared to layering processed which are used to form more traditional absorbent structures, such as absorbent structure 1030. Thus, the reinforcing web material 329 can maintain a high-level of void volume, even after many insults-since superabsorbent material 317 which has penetrated the reinforcing web material 329 will be swollen after a first and/or second insult, thereby blocking off void volume of the reinforcing web material 329. This result helps to ensure the absorbent structures of the present disclosure maintain a good intake when handling multiple liquid insults.

FIGS. 10A-10C depict different cross-sections of exemplary absorbent structures 101A-101C as formed by process 300, according to aspects of the present disclosure. The cross-sections representing FIGS. 10A-10C are taken along line 10-10 of FIG. 7, showing different configurations of the mixture 320, the web material 303, the reinforcing web material 329, and the web material 324, where present.

FIG. 10A depicts an embodiment of an absorbent structure 101A of the present disclosure comprising web material 303, reinforcing web material 329, and web material 324, with mixture 320 disposed between web material 303 and the reinforcing web material 329. The web material 303 and web material 324 may have top surfaces 342 and 344, respectively, and bottom surfaces 343 and 345, respectively. In some exemplary embodiments according to FIG. 10A, the mixture 320 may be disposed on top surface 342 of web material 303 with the reinforcing web material 329 disposed mixture 320 and the bottom surface 345 of the web material 324. In some of these embodiments, absorbent structure 101A may further comprise seaming adhesives 346 disposed outboard of the mixture 320 and bonding the bottom surface 345 of web material 324 to the top surface 342 of web material 303. Such seaming adhesives 346 may help to seal the side edges 358a, 358b of the absorbent structure 101A closed. However, it should be understood that such adhesives 346 are not necessary in all embodiments-many embodiments sufficiently capture the superabsorbent material 317 such that little to no superabsorbent material 317 may escape out of the absorbent structure 101A, even without the seaming adhesive 346.

Where present, the seaming adhesives 346 may be applied by adhesive applicators prior to or after deposition of the mixture 320 (for example, optional adhesive applicators 305 and/or 325 may apply the seaming adhesives 346). Alternatively, the seaming adhesives 346 may be applied during deposition of the mixture 320 by adhesive applicators 307, 309, 333 and/or 335, for example where the adhesive spray from the adhesive applicators 307, 309, 333 and/or 335 is wider than the stream or streams of superabsorbent material 317 (e.g. in the cross-machine direction 338 of process 300). In other embodiments, however, absorbent structure 101 may not include any seaming adhesives 346. In such embodiments the adhesives 308, 310, 334, and/or 336 may be sufficient to bond the web material 303 to the web material 324.

FIG. 10B depicts another embodiment of an absorbent structure 101B of the present disclosure comprising web material 303 and web material 324, with mixture 320 disposed between web material 303 and web material 324. In this embodiment, in contrast to the embodiment of FIG. 10A, instead of the bottom surface 345 of web material 324 bonded to the top surface 342 of web material 303, the bottom surface 345 of web material 324 may be bonded to the bottom surface 343 of web material 303. For example, the web material 324 may wrap at least partially around the mixture 320, sometimes termed a C-wrap, such that the bottom surface 345 of web material 324 is disposed about both a portion of a first side of the mixture 320 and a second side of the mixture 320. In the embodiment shown in FIG. 10B, the web material 303 may be disposed between the mixture 320 and the web material 324 where the web material 324 and the web material 303 overlap. Although, in other embodiments, the web material 324 may wrap around the mixture 320 and be disposed between the mixture 320 and the web material 303 where the web material 324 and the web material 303 overlap.

In the embodiment shown in FIG. 10B, the absorbent structure 101B may include seaming adhesives 346 connecting the bottom surface 345 of web material 324 to the bottom surface 343 of web material 303 proximate lateral edges of the absorbent structure 101B. Although, it should be understood that such seaming adhesives 346 are optional and may not be present in all embodiments. Where present, seaming adhesives 346 may be applied for example, by optional adhesive applicators 305 and/or 325, or may be applied by one or more of adhesive applicators 307, 309, 333 and/or 335.

FIG. 10C depicts another embodiment of an absorbent structure 101C of the present disclosure comprising only web material 303 and not web material 324. In this embodiment, web material 303 wraps around the mixture 320, for example forming a C-wrap configuration. As shown in FIG. 10C, the web material 303 has web end portions 347 and 349. In some exemplary embodiments according to FIG. 10C, the web material 303 may wrap around the mixture 320 such that the web end portions 347 and 349 overlap each other. Such configurations may further include one or more seaming adhesives 346 disposed between the web end portions 347 and 349 and bonding the web end portions 347 and 349 of the material 303 together. Although, such seaming adhesives 346 are optional and may not be present in other embodiments. In further embodiments according to FIG. 10C, the web end portions 347 and 349 may be spaced from each other such that the web end portions 347 and 349 do not overlap. In such embodiments, a portion of the mixture 320 may be left uncovered by the web material 303.

With respect to FIGS. 10A-10C, the exemplary absorbent structures 101A-101C may have top sides 362 and bottom sides 364. However, it should be understood that these absorbent structures 101A-101C may be used in any orientation. For example, in some instances, the described absorbent structures 101A-101C may be placed into an absorbent article, such as article 10, with the top side 362 disposed most closely to the body facing surface 19. In other instances, the absorbent structures 101A-101C may be placed into an absorbent article such as article 10 with the bottom side 364 disposed most closely to the body facing surface 19.

Where web material 303 forms the top side 362 of the absorbent structures 101A-101C and where the top side 362 is disposed most closely to the body facing surface 19, the web material 303 may be any suitable nonwoven material—for instance, a bonded carded web, a meltblown material, a spunbond material, including spunbond and meltblown combination webs commonly referred to SMS webs or SMMS webs or the like, a spunlace material, a hydroentangled material, an airlaid material, a coform material, or may be a material formed according to mixtures of technologies used to form the above described materials such as a spunbond-meltblown-spunbond material or other such similar materials. Typical basis weights for such web materials 303 may range between 8 gsm to 200 gsm, or between 10 gsm and 150 gsm, or between 10 gsm and 100 gsm. Alternatively, the web material 303 may be formed of wetlaid fibrous materials such as uncreped through air dried tissue or creped tissue, or other material sheets made from cellulosic fibers. The web material 303 may further comprise a combination of nonwoven and fibrous materials, including fiberized pulp captured on top of or between nonwoven materials or wetlaid fibrous materials. In such embodiments, the fiberized pulp may be densified to form the web material 303—prior to its use in capturing superabsorbent 317 and adhesive 308 and/or 310.

Without respect to any specific type of material, it has been found that web material 303 should ideally have sufficient air permeability to allow vacuum air flow to pass through the web material 303 and to at least partially entrain the streams 319 (and optionally 331) of superabsorbent material 317 and adhesives 308 and/or 310 (and optionally 334 and/or 336) in such vacuum air flow. For example, it has been found that an air permeability of the web material 303 should be greater than 25 standard cubic feet per minute (SCFM) of air (0.71 standard cubic meters per minute (SCMM). In further embodiments, it may be more preferable for the web material 303 to have an air permeability of greater than 50 SCFM (1.4 SCMM), or greater than 75 SCFM (2.1 SCMM). Such measurements of air permeability may be made consistent with standard industry practices for measuring air permeability. According to some examples, such air permeability measurements may be made with a Frazier Instruments Model LP air permeability tester from Frazier Instruments (offices in Hagerstown, Maryland), a Textest FX 3300 air permeability unit from Textest (offices in Schwerzenbach, Switzerland), or equivalent test unit.

Again, where the web material 303 forms the top side 362 of the absorbent structures 101A-101C and where the top side 362 is disposed most closely to the body facing surface 19 it may be preferable that the fibers, or at least the surface fibers, of the web material 303 have sufficient wettability to allow fluid intake, fluid flow, and fluid distribution through the web material 303 to the superabsorbent material 317. In some embodiments, the wettability may come from the composition of the fiber. For instance, the fibers forming the web material 303 may be inherently wettable fibers include such as natural cellulosic fibers derived from cotton, wood, or other fibers. Other examples of an inherently wettable fiber include a reconstituted cellulosic fiber such as a rayon fiber. In further embodiments, the fibers forming the web material 303 may not be inherently wettable, but may be changed to be wettable, such as by addition of a surfactant treatment to the fibers, or at least to the surface fibers. The surfactant treatment may be applied at least to the surface fibers in a continuous or discontinuous manner. In other embodiments, a surfactant treatment can be added internally to the fiber which will ultimately migrate to the surface of the fiber.

Where web material 324 forms the bottom side 364 of the absorbent structures 101A-101C and where the bottom side 364 is disposed most closely to the body facing surface 19, the web material 324 may be any suitable nonwoven material—for example, any of those recited with respect to web material 303. Additionally, the web material 324 may be preferred to have any of the same properties as were described above with respect to web material 303. Where web material 324 forms the bottom side 364 of the absorbent structures 101A-101C and where the top side 362 is disposed most closely to the body facing surface 19, the web material 324 may also be any of the materials described above with respect to web material 303, including having any of the properties and their described ranges.

The adhesives 308 and/or 310 may generally comprise hot-melt adhesives, and the nozzles 321, 323 may be configured to project the adhesives 308 and/or 310 toward the stream 319 of superabsorbent material 317 such that the adhesives 308 and/or 310 form adhesive filaments 316. Desirably, the adhesives 308 and/or 310 should have sufficient tack and cohesion. An exemplary suitable adhesives are the TECHNOMELT DM 5402U adhesive available from Henkel Corporation, a company having offices in Rocky Hill, Connecticut, the FOCUS T137 or FOCUS T193 adhesive available from Focus Hotmelt Co Ltd, a company with offices at 15F Development Center Mansion 3 Linjiang Avenue Tianhe Distr Guangzhou, 510623 China. This suitable adhesive is a styrenic block copolymer based hot melt adhesive design to have high cohesion and strong specific adhesion to provide good fixation of the superabsorbent material 317 in the absorbent structure under both wet and dry conditions. It may further be generally preferable for the adhesives 308 and/or 310 to be non-water soluble in order to help retain the positioning of the superabsorbent material 317 within the structure 101 after one or more liquid insults. It has been found that rubber-based adhesives may be preferable in that they may produce structures 101 which perform superior to other adhesives, such as standard construction adhesives or olefin-based adhesives.

In general, the adhesive applicators 307 and/or 309 operate to spray the adhesives 308 and/or 310 such that the adhesives 308 and/or 310 form adhesive filaments 316 which contact the stream 319. The adhesive applicators 307 and/or 309 may be generally configured to spray the adhesives 308 and/or 310 such that the adhesives 308 and/or 310 form filaments 316 having preferred diameters. It has been found that it may be preferable for the filaments 316 to have diameters of between 25 micrometers (microns) and 150 microns, or between 50 microns and 100 microns, or between 75 microns and 100 microns. These ranges of filament diameters have been shown to work together well with superabsorbent material 317 having the below described particle diameters in order to provide for beneficial performance properties of the structures 101.

Although the above described adhesive properties have been described with respect to adhesives 308 and/or 310, where present the adhesives 334 and/or 336 may have properties similar to those described above with respect to adhesives 308 and/or 310. Likewise, where present, the applicators 333 and/or 335 may be configured to spray the adhesives 334 and/or 336 in a similar manner to how the adhesive applicators 307 and/or 309 are configured to spray the adhesives 308 and/or 310. For example, the diameters of the filaments 316 formed by the adhesives 334 and/or 336 being sprayed from applicators 333 and/or 335 may be similar to the diameters described above with respect to filaments 316 formed by the adhesives 308 and/or 310 being sprayed from applicators 307 and/or 309.

Additionally, it has been found that the sizes of the individual particles 318 of the superabsorbent material 317 may drive certain desired properties of the formed absorbent structures 101. For instance, particle size of the individual particles 318 may at least partially drive pad integrity and superabsorbent material capture values, particularly in conjunction with the described structural features of the adhesive filaments 316. For example, it has been found that where the bulk superabsorbent material 317 has mean particle sizes ranging from between 150 and 1000 micrometers (microns) in diameter provide good results-especially in conjunction with the above-described adhesive filament 316 diameters. In such embodiments, it may be preferred that at least 50% of the mass of the bulk superabsorbent material 317 have diameters larger than 180 microns. In other embodiments, it may be preferred that at least 60%, or at least 70%, or at least 80% of the mass of the bulk superabsorbent material 317 have diameters larger than 180 microns. In further embodiments, it may more preferable that at least 50% of the mass of the bulk superabsorbent material 317 have diameters larger than 300 microns, or that at least 60%, or at least 70%, or at least 80% of the mass of the bulk superabsorbent material 317 have diameters larger than 300 microns.

Where the bulk superabsorbent material 317 mean particle size is too low, such as lower than 300 microns, or lower than 180 microns, the formation and performance of the structures 101 can be affected to a detrimental degree. For example, such small mean particle sizes may affect the ability of the superabsorbent material 317 to fall in a relatively uniform stream from the chute 315, thereby resulting in relatively more non-uniformity of the superabsorbent material 317 and adhesives 308 and/or 310 (and optionally 334 and/or 336). Additionally, such small average particle sizes may begin to approach the average diameter size of the adhesive filaments 316, both affecting capture of the individual particles 318 by the adhesive filaments 316 and reducing absorptive performance because the adhesive filaments 316 would more readily block liquid from accessing all portions of the individual particles 318. Determining the masses of different portions of particles of the bulk superabsorbent material 317 may be performed by any classification process known in the art. For example, it is well known to utilize multiple sieves with differing mesh sizes to separate out different portions of particles from the bulk superabsorbent material 317 having differing particle size diameters. One particular method which can be used in such a classification effort may be ASTM D1921-18, titled “Standard Test Methods for Particle Size (Sieve Analysis) of Plastic Materials”.

In the embodiments of absorbent structures 101A-101C, it may be particularly preferred for the mixture 320 to comprise superabsorbent material 317 in amounts between about 100 gsm and about 700 gsm, or more particularly between about 150 gsm and about 700 gsm, or between about 200 gsm and about 700 gsm, or between about 250 gsm and about 700 gsm, or between about 250 gsm and about 600 gsm, or between about 250 gsm and about 550 gsm. In these embodiments, the adhesives 308 and/or 310 may combine to equal an add-on percentage of between about 2% and about 7%, or more particularly between about 2% and about 6%, or between about 2% and about 5.5%, or between about 2% and about 5%, or between about 2% and about 4.5%, or between about 2.5% and about 4.5%, or between about 2.5% and about 4%. It may be particularly advantageous for the adhesives 308 and/or 310 to combine to equal an add-on percentage of less than about 5%, less than about 4.5%, or less than about 4%, or less than about 3.5%, or less than about 3%, or less than about 2.5%. The add-on percentage, in this context, is the amount of adhesive relative to the amount of superabsorbent material 317. Accordingly, as one example, an absorbent structure 101A having an absorbent material 317 basis weight of 500 gsm and an adhesive add-on percentage of 5% would result in the adhesive being present in an amount of about 25 gsm (5% of 500 gsm).

In the above embodiments of structures 101 according to FIGS. 10A-10C, it may be particularly advantageous for the web materials 303, 324 to be hydrophobic and to be a type of web material such as a TABCW, spunlace, or spunbond materials having basis weights between about 10 gsm and about 80 gsm. In particular, such web materials 303, 324 may be formed of polypropylene (PP) fibers, polyethylene (PE) fibers, bicomponent fibers comprising both PP and PE material or PE and polyethylene terephthalate (PET) material, Rayon fibers, and pulp fibers, or any combinations of such materials.

The reinforcing web material 329 may be preferred to be a non-woven material comprised of multiple individual fibers such as a spunbond material or a spunbond-meltblown-spunbond (SMS) material. In other embodiments, the nonwoven material may be a porous nonwoven material such as a through-air bonded carded web (TABCW) or chemically bonded nonwoven materials or the like. In still further embodiments, the nonwoven material may be a spunlace material, including combinations of TABCW materials with Rayon fibers or SMS materials with Rayon fibers. In still further embodiments, the reinforcing web material 329 may be comprised substantially of polyolefin bi-component fibers, or polyolefin mixed bi-component and eccentric fibers, or just polyolefin eccentric fibers. In some specific preferred examples, a TABCW material may be used comprising between 60% to 80%, by weight, eccentric bi-component fibers (comprising polyethylene and polypropylene) having a denier of between about 3 and about 7 and between 20% and 40%, by weight, non-eccentric bi-component fibers (comprising polyethylene and polypropylene) having a denier of between about 1 and about 3. Although, it should be understood that these are just some exemplary materials. Other suitable materials may be used in other contemplated embodiments.

The reinforcing web material 329 may have a preferred basis weight between about 10 gsm to about 120 gsm. In more specific embodiments, the reinforcing web material 329 may have a basis weight of between about 10 gsm to about 100 gsm, or between about 10 gsm to about 90 gsm, or between about 10 gsm to about 80 gsm, or between about 10 gsm to about 70 gsm, or between about 20 gsm to about 70 gsm, or between about 30 gsm to about 70 gsm, or any other suitable basis weight.

FIGS. 11A-11C depict different cross-sections of exemplary absorbent structures 101A′-101C′ as formed by process 400, according to aspects of the present disclosure. The cross-sections representing FIGS. 11A-11C are taken along line 11-11 of FIG. 9, showing different configurations of the mixtures of superabsorbent material 317 and adhesives 308, 310, 334, and/or 336, the web material 303, the reinforcing web material 329, and the web material 324, where present.

FIG. 11A depicts an embodiment of an absorbent structure 101A′ of the present disclosure comprising web material 303, reinforcing web material 329, and web material 324, with mixtures 320a and 320b disposed between web material 303 and the web material 324. The web material 303 and web material 324 may have top surfaces 342 and 344, respectively, and bottom surfaces 343 and 345, respectively. In some exemplary embodiments according to FIG. 11A, the mixture 320a may be disposed on top surface 342 of web material 303 with the reinforcing web material 329 disposed between the mixture 320a and the web material 324. The mixture 320b may be disposed on the bottom surface 345 of the web material 324, with the reinforcing web material 329 disposed between the mixture 320b and the web material 303. In such embodiments, the reinforcing web material 329 is disposed directly between the mixture 320a and the mixture 320b.

In some of these embodiments, absorbent structure 101A′ may further comprise seaming adhesives 346 disposed outboard of the mixtures 320a, 320b and bonding the bottom surface 345 of web material 324 to the top surface 342 of web material 303. Such seaming adhesives 346 may help to seal the side edges 358a, 358b of the absorbent structure 101A′ closed. However, it should be understood that such adhesives 346 are not necessary in all embodiments-many embodiments sufficiently capture the superabsorbent material 317 such that little to no superabsorbent material 317 may escape out of the absorbent structure 101A′, even without the seaming adhesive 346.

Where present, the seaming adhesives 346 may be applied by adhesive applicators prior to or after deposition of the mixtures 320a, 320b (for example, optional adhesive applicators 305 and/or 325 may apply the seaming adhesives 346). Alternatively, the seaming adhesives 346 may be applied during deposition of the mixtures 320a, 320b by adhesive applicators 307, 309, 333 and/or 335, for example where the adhesive spray from the adhesive applicators 307, 309, 333 and/or 335 is wider than the stream or streams of superabsorbent material 317 (e.g. in the cross-machine direction 338 of process 300). In other embodiments, however, absorbent structure 101A′ may not include any seaming adhesives 346. In such embodiments the adhesives 308, 310, 334, and/or 336 may be sufficient to bond the web material 303 to the web material 324.

FIG. 11B depicts another embodiment of an absorbent structure 101B′ of the present disclosure comprising web material 303 and web material 324, with mixtures 320a, 320b disposed between web material 303 and web material 324—with the reinforcing web material 329 disposed directly between the mixtures 320a, 320b. In this embodiment, in contrast to the embodiment of FIG. 11A, instead of the bottom surface 345 of web material 324 bonded to the top surface 342 of web material 303, the bottom surface 345 of web material 324 may be bonded to the bottom surface 343 of web material 303. For example, the web material 324 may wrap at least partially around the mixture 320, sometimes termed a C-wrap, such that the bottom surface 345 of web material 324 is disposed about both a portion of a first side of the mixture 320 and a second side of the mixture 320. In the embodiment shown in FIG. 11B, the web material 303 may be disposed between the mixture 320 and the web material 324 where the web material 324 and the web material 303 overlap. Although, in other embodiments, the web material 324 may wrap around the mixture 320 and be disposed between the mixture 320 and the web material 303 where the web material 324 and the web material 303 overlap.

In the embodiment shown in FIG. 11B, the absorbent structure 101B′ may include seaming adhesives 346 connecting the bottom surface 345 of web material 324 to the bottom surface 343 of web material 303 proximate lateral edges of the absorbent structure 101B′. Although, it should be understood that such seaming adhesives 346 are optional and may not be present in all embodiments. Where present, seaming adhesives 346 may be applied for example, by optional adhesive applicators 305 and/or 325, or may be applied by one or more of adhesive applicators 307, 309, 333 and/or 335.

FIG. 11C depicts another embodiment of an absorbent structure 101C′ of the present disclosure comprising only web material 303 and not web material 324. In this embodiment, web material 303 wraps around the mixture 320, for example forming a C-wrap configuration. As shown in FIG. 11C, the web material 303 has web end portions 347 and 349. In some exemplary embodiments according to FIG. 11C, the web material 303 may wrap around the mixture 320 such that the web end portions 347 and 349 overlap each other. Such configurations may further include one or more seaming adhesives 346 disposed between the web end portions 347 and 349 and bonding the web end portions 347 and 349 of the material 303 together. Although, such seaming adhesives 346 are optional and may not be present in other embodiments. In further embodiments according to FIG. 11C, the web end portions 347 and 349 may be spaced from each other such that the web end portions 347 and 349 do not overlap. In such embodiments, a portion of the mixture 320 may be left uncovered by the web material 303.

With respect to FIGS. 11A-11C, the exemplary absorbent structures 101A′-101C′ may have top sides 362 and bottom sides 364. However, it should be understood that these absorbent structures 101A′-101C′ may be used in any orientation. For example, in some instances, the described absorbent structures 101A′-101C′ may be placed into an absorbent article, such as article 10, with the top side 362 disposed most closely to the body facing surface 19. In other instances, the absorbent structures 101A′-101C′ may be placed into an absorbent article such as article 10 with the bottom side 364 disposed most closely to the body facing surface 19.

In the embodiments of absorbent structures 101A′-101C′, it may be particularly preferred for the mixtures 320a, 320b to each comprise superabsorbent material 317 in amounts between about 50 gsm and about 350 gsm, or more particularly between about 50 gsm and about 300 gsm, or between about 50 gsm and about 275 gsm, or between about 75 gsm and about 275 gsm, or between about 100 gsm and about 275 gsm, or between about 100 gsm and about 250 gsm. In these embodiments, the adhesives 308 and/or 310 in the mixture 320a and the adhesives 334 and/or 336 in the mixture 320b may combine to equal an add-on percentage of between about 2% and about 7%, or more particularly between about 2% and about 6%, or between about 2% and about 5.5%, or between about 2% and about 5%, or between about 2% and about 4.5%, or between about 2.5% and about 4.5%, or between about 2.5% and about 4% within each of the mixtures 320a, 320b. It may be particularly advantageous for the adhesives 308 and/or 310 within the mixture 320a and the adhesives 334, 336 within the mixture 320b to combine to equal an add-on percentage of less than about 5%, less than about 4.5%, or less than about 4%, or less than about 3.5%, or less than about 3%, or less than about 2.5%.

Further in the embodiments of absorbent structures 101A′-101C′, the particularly advantageous materials and basis weights for the web materials 303, 324 may be the same as the materials described above with respect to absorbent structures 101A-101C. Similarly, the particularly advantageous materials and basis weights for the reinforcing web material 329 of the absorbent structures 101A′-101C′ may be the same as those described above with respect to the absorbent structures 101A-101C. The preferred void volumes for the reinforcing web material 329 in the embodiments of absorbent structures 101A′-101C′ may be the same general ranges as described above for the absorbent structures 101A-101C.

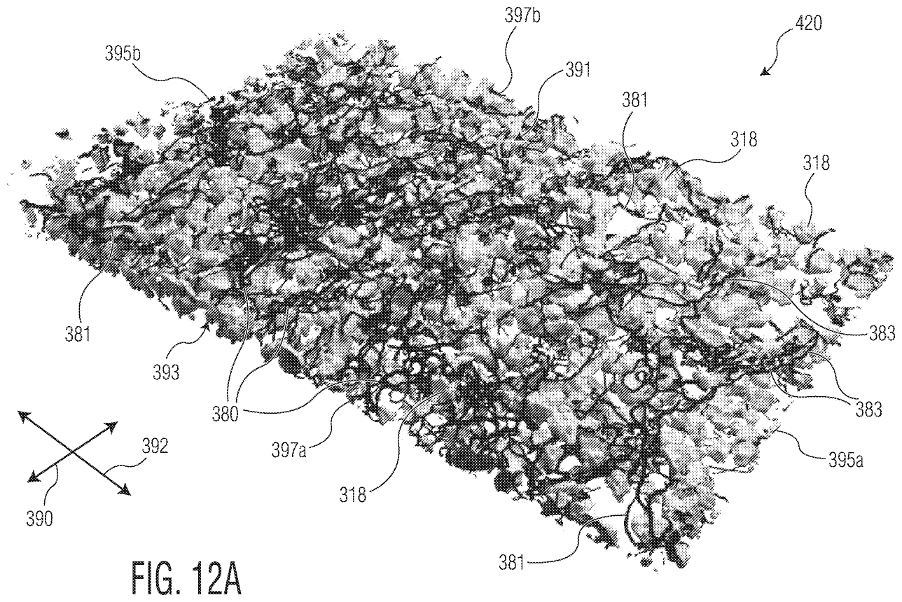

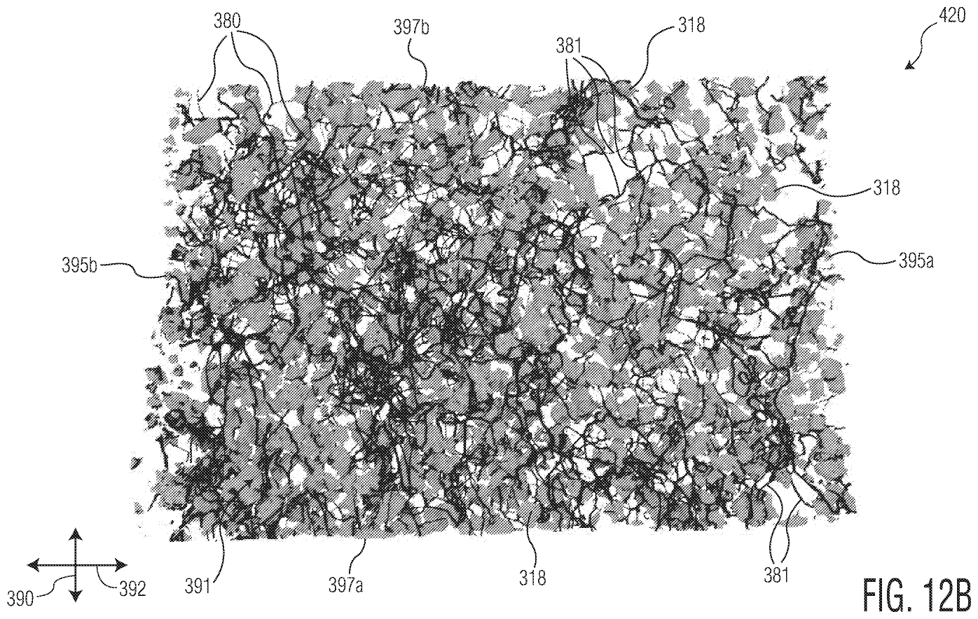

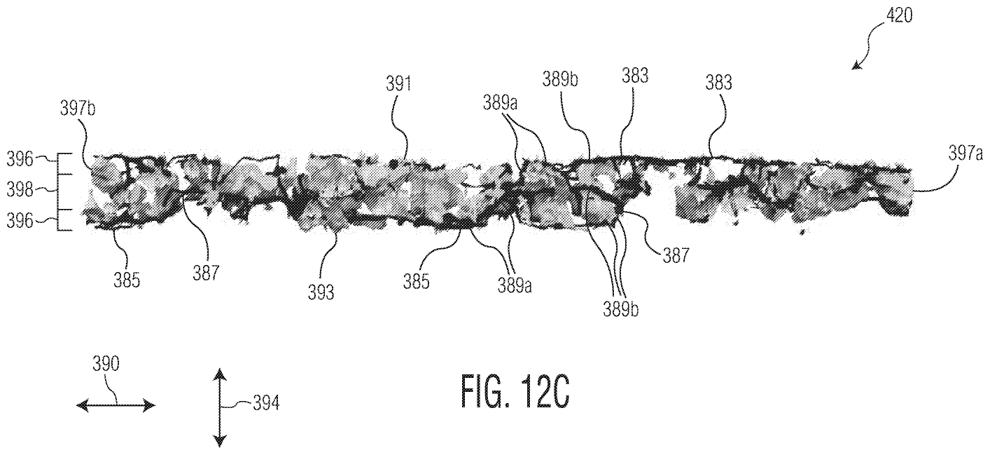

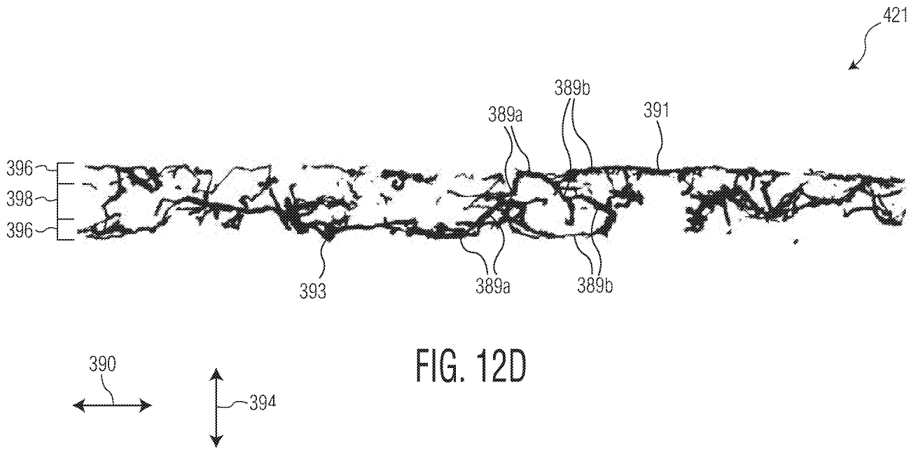

FIG. 12A is a perspective view of a computer-generated image 420 of a deposited mixture 320 which is based on micro-CT images taken from an exemplary deposited mixture 320 formed by the process 300. More specifically, the mixture 320 used to generate the computer-generated mixture 420 shown in FIG. 12A was formed by process 300, including the details of the absorbent deposition station 302 of FIG. 8, where the stream 319 and the adhesives 308 and 310 were configured to be same as listed for the first exemplary absorbent structures detailed below, and the resulting structure 320 had superabsorbent material 317 disposed in an amount of 400 gsm and where the adhesives 308, 310, 334, and 336 were present at an add-on rate of 5%. The mixture 320 was stained with osmium tetroxide and then micro-CT scanning was performed, both according to standard, known techniques for staining and scanning. As part of the micro-CT process, a portion of stained deposited mixture 320 chosen approximately from the center of the mixture 320 (e.g. structure 101) in the widthwise and lengthwise directions was chosen for imaging. The portion had dimensions of approximately 3 cm by 1 cm and was sliced into approximately one-thousand two-hundred and fifty individual segments extending in the lateral direction 392, each segment extending from end edge 395a to end edge 395b and comprising 1986 pixels in the longitudinal dimension (e.g. along longitudinal direction 392). Each segment further comprised 504 pixels in the vertical direction 394 between the first surface 391 and the second surface 393. A voxel size of 8.0 micrometers was used. From the captured segments, a three-dimensional model was generated and is depicted in FIGS. 12A-12D

The adhesive filaments 316 which were sprayed by the applicators 307, 309, 311 and/or 313 cross and connect as the adhesives 308, 310, 334, and/or 336 and the superabsorbent material 317 intermix to form a three-dimensional mesh network 380 having network adhesive filaments 381 extending substantially throughout the three-dimensional space formed by image 420, as can be seen in FIGS. 12A and 12B. The network adhesive filaments 381 extend or are oriented in a substantially random manner throughout the three-dimensional mesh network 380—likely due to the random, turbulent nature of the mixing of the adhesives 308, 310, 334, and/or 336 and the superabsorbent material 317 prior to deposition. As used herein, the network adhesive filaments 381 may be considered to extend substantially throughout the three-dimensional space formed by the deposited mixture of the image 420 where the network adhesive filaments 381 extend between and intermingle with a majority, or super-majority, of the individual superabsorbent particles 318. Such a configuration is in contrast to configurations where adhesive filaments extend over pockets or groupings of superabsorbent particles and do not extend into and between individual superabsorbent particles 318 of the pocket or grouping of superabsorbent particles 318 and/or where adhesive filaments are applied to deposited superabsorbent material 317—particularly where the adhesive filaments are applied in a pattern (e.g. a spray, swirl, line pattern, or the like). The superabsorbent material 317 are also disposed throughout the three-dimensional mesh network 380, shown as particles 318, and are immobilized by contact with one or more of the network adhesive filaments 381.

The processes 300 and 400 may operate to intermix the adhesives 308, 310, 334, and/or 336 with the superabsorbent material 317 to such a degree that the network adhesive filaments 381 contact substantially all of the individual superabsorbent material 317. The network adhesive filaments 381 may wrap around a majority, or a super-majority, of the individual superabsorbent particles 318. As used herein, the network adhesive filaments 381 may be considered to wrap around an individual superabsorbent particle 318 if combined lengths of individual network adhesive filaments 381 in contact with an individual superabsorbent particle 318 equal at least 40% of a maximum circumference of the individual superabsorbent particle 318.

As seen in both FIG. 12A and FIG. 12B, which is a top plan view of a portion of the image 420 of FIG. 12A, along with FIG. 12C, the image 420 of the deposited mixture may generally have a first surface 391 and a second surface 393 disposed opposite the first surface 391, along with end edges 395a, 395b and side edges 397a, 397b. Each of the first surface 391 and the second surface 393 extend generally in the lateral and longitudinal directions 390, 392. At each of the first surface 391 and the second surface 393, the mesh network 380 may comprise network adhesive filaments 381 which extend substantially in the lateral and longitudinal directions 390, 392. For example, first network adhesive filaments 383 can be seen extending substantially in the lateral and longitudinal directions 390, 392 along the first surface 391. Second network adhesive filaments 385 (shown in FIG. 12C) may extend substantially in the lateral and longitudinal directions 390, 392 along the second surface 393.

The network adhesive filaments 381 of the three-dimensional mesh network 380 may further include vertically extending filaments 387 which can be seen extending in the vertical direction 394 in FIG. 12C. FIG. 12C represents a laterally extending slice of the image 420 of FIG. 12A and having a length in the longitudinal direction 392 of 0.5 mm, showing in more detail interaction of the particles 318 and adhesive filaments 381. FIG. 12D is the same image as FIG. 12C with the particles 318 removed to show in more detail the adhesive filaments 381 and their disposition through the vertical direction 394.

At least some of these vertically extending filaments 387 extend all the way from the first surface 391 to the second surface 393 and connect the first network adhesive filaments 383 to the second network adhesive filaments 385 to form the three-dimensional mesh network 380. Of course, as can be seen, the vertically extending filaments 387 may not extend perfectly in the vertical direction 394 and may twist and turn between and around individual superabsorbent particles 318 such that at least some of the vertically extending filaments 387 extend also in the lateral and/or longitudinal directions 390, 392. In at least some embodiments, individual network adhesive filaments 381 may themselves extend from along part of the first surface 391 (for example in the longitudinal and/or lateral directions 390, 392), transition to extending in the vertical direction 394, and then connect with the second surface 392—possibly further extending along the longitudinal and/or lateral directions 390, 392 at the second surface 392. Such behavior can be seen with respect to network adhesive filaments 389a and 389b.

Another feature that can be seen to some extent in the FIGS. 12C-12D is the relative distribution of the network adhesive filaments 381 within different vertical regions of the deposited mixture represented by the image 420. For example, as indicated in FIGS. 12C and 12D, the image 420 may be split into exterior regions 396 and an interior region 398 disposed between the exterior regions 396, spanning the vertical direction 394. The exterior regions 396 may each be defined by a thickness of 33% of the overall thickness of the structure 420, while the interior region 398 may be defined by a thickness of 33% of the overall thickness of the structure 420.

It has been found that the processes 300 and/or 400 may desirably penetrate the adhesives 308, 310, 334, and/or 336 into the interior region 398, thereby promoting high superabsorbent particle capture values and greater pad uniformity through greater evenness of distribution of the superabsorbent material 317 and adhesives 308, 310, 334, and/or 336 throughout the structure 420. This is particularly true where the formed mixtures 320 of the present disclosure have basis weights of superabsorbent material 317 greater than 300 gsm, or greater than 400 gsm, or greater than 500 gsm, or greater than 600 gsm, or greater than 700 gsm. As the desired basis weights of superabsorbent material 317 in a mixture 320 increases, penetration of the adhesives 308, 310, 334, and/or 336 into an interior of a streams 319 and/or 331 becomes more difficult and where the processes 300 and/or 400 excel in comparison to prior art processes.

In order to assess the ability of the processes 300 and/or 400 to penetrate adhesive into the interior region 398 of the formed mixtures 320, analysis of two sample codes was performed. In the analysis, two sample codes were generated according to process 400 having a basis weight of superabsorbent material of 500 gsm and adhesive disposed in an add-on amount of 5%. From these two sample codes, micro-CT images of portions of the codes were formed-according to the standard processes and techniques mentioned above. The Binder Distribution Test Method, described in detail below, was then performed on the generated micro-CT images to determine the relative quantity of adhesive located within the interior region 398 of the imaged portion of the two sample codes. The micro-CT images were generated using the known staining and imaging methods described above.

According to the Binder Distribution Test Method, it was found that the first sample code had 28.0% of the of the total amount of adhesive within the first sample code located within the interior region 398 of the first sample code, with the standard deviation being 8.4%. The second sample code was found to have 30.7% of the total amount of adhesive within the second sample code located within the interior region 398 of the second sample code, with the standard deviation being 8.9%. Accordingly, mixtures 320 formed according to the processes 300 and/or 400 can cause greater than 28% of the total amount of adhesive within the mixture 320 to be located within the interior region 398, or greater than 30.5% of the total amount of adhesive within a mixture 320 to be located within the interior region 398. However, in further potential embodiments, it is believed that greater than 33%, or even greater than 35% of the total amount of adhesive within a mixture 320 being located within the interior region 398 of the mixture 320 can be achieved through slight modifications to the processes 300 and/or 400—for example in terms of adhesive add-on amount, vacuum energy, nip pressure, location and angle of the nozzles, and the like. Such high adhesive penetration into the interior region 398 of the formed mixtures of the processes 300 and/or 400 result help to drive improved absorbent structure properties including the capture and stabilization of the superabsorbent particles (e.g. resulting in less ‘free’ particles), pad integrity-particularly wet pad integrity—and even a relative uniformity of the distribution of the superabsorbent particles within the absorbent structures 101, 101′.

The absorbent structures 101, 101′ produced by the process 300 and/or 400 have been shown to have beneficial characteristics with respect to prior art absorbent structures. For example, the processes 300 and/or 400 have been shown to produce absorbent structures 101, 101′ providing superior performance with respect to the capture and immobilization of superabsorbent material 317, as well as a superior liquid intake ability, particularly with respect to 3rd intake times, as will be described in more detail below.

In order to compare absorbent structures, a number of different absorbent structures 101′ were formed by the described process 400 and tested with respect to absorbent structures formed by prior art processes, such as prior art absorbent structure 1030. As will be described below, exemplary absorbent structures 101′ and exemplary prior art absorbent structures 1030 were compared with respect to the Cradle Intake Time Test Method, the SAM Penetration Test Method, and the Free SAM Test Method, as described herein below, to produce comparative results.

### First Exemplary Absorbent Structures

First exemplary absorbent structures 101′, labeled as Example 1 herein, were formed according to the exemplary process 400. Specifically, first exemplary absorbent structures 101′ were formed according to the exemplary process 400 having superabsorbent particulate basis weights of 320 gsm; with 160 gsm of superabsorbent particulates applied within mixture 320a, and 160 gsm of superabsorbent particulates applied within mixture 320b. The adhesive add-on within each of the mixtures 320a, 320b was approximately 2.85%.

The settings of the process 400 used to form the first exemplary absorbent structures 101′ include using both adhesive applicators 307, 309 with adhesive applicator 307 positioned a distance from the web material 303 and the stream 319 such that the adhesive contacted the stream 319 a distance of 6.4 mm from the web material 303 (e.g. the distance 361). The adhesive applicator 309 was positioned a distance from the web material 303 and the stream 319 such that the adhesive 310 contacted the stream 319 a distance of 16 mm from the web material 303 (e.g. the distance 367 plus the distance 361). Additionally, the chute 315 was positioned a distance 359 of 76 mm from the web material 303. The adhesive nozzle 321 was positioned at an angle 359a of 60 degrees with respect to the machine direction 330, and the adhesive nozzle 323 was also positioned at an angle of 60 degrees with respect to the machine direction 330. The nozzles 321 and 232 were Universal™ Signature™ Spray Nozzles available from the Nordson Corporation, utilizing a Focus T137 adhesive. The chute width 356 was set at 12 mm, and a nip pressure at nip station 327 was 1 PLI (175.1 N/m). An 8 gsm SMS material was used for material web materials 303 and 324, and a 40 gsm Spunlace material, which combined a TABCW material with Rayon fiber, was used for the reinforcing web material 329. Vacuum energy was applied such that the forming surface had a pressure differential of approximately 0.51 m of water.

### Second Exemplary Absorbent Structures

The second exemplary absorbent structures are substantially the same as the Example 1 structures, except that the adhesive add-on within each of the mixtures 320a, 320b was approximately 3.56%. The second exemplary absorbent structures are labeled as Example 2 herein.

### Third Exemplary Absorbent Structures

The third exemplary absorbent structures are substantially the same as the Example 1 structures, except that the adhesive add-on within each of the mixtures 320a, 320b was approximately 1.44%. The third exemplary absorbent structures are labeled as Example 3 herein.

### Comparative Absorbent Structures

Comparative absorbent structures 1030, labeled as Comparative Example 1 herein, were formed according to a prior art layering process. A first adhesive was applied to a first web material, a first layer of superabsorbent particles was applied to the first adhesive, and a second adhesive was applied to the first layer of superabsorbent particles. Next, a reinforcing web material was applied to the second adhesive. The absorbent layering process was repeated with a third adhesive being applied to the reinforcing web material, a second layer of superabsorbent particles was applied to the third adhesive, and a fourth adhesive was applied to the second layer of superabsorbent particles. Finally, a second web material was applied to the fourth adhesive.

The comparative absorbent structures 1030 were formed having superabsorbent particulate basis weights of 320 gsm; with 160 gsm of the superabsorbent particulates applied within the first layer of layer of superabsorbent particles, and 160 gsm of the superabsorbent particulates applied within the second layer of superabsorbent particles. The adhesive was applied at an add-on percentage of 5%, split approximately equally between the first through fourth adhesives. An 8 gsm SMS material was used for the top facing material layer 1042 and the bottom facing material layer 1044, and a 40 gsm Spunlace material, which combined a TABCW material with Rayon fiber, was used for the internal reinforcing material 1046. Vacuum energy was applied such that the forming surface had a pressure differential of approximately 0.20 m of water and the structures 1030 were nipped at pressure of 1 PLI (175.1 N/m).

For the comparative results according to the Cradle Intake Test Method described below, the Example 1 and Comparative Example 1 absorbent structures were placed into identical absorbent garment products.

### Free SAM Determination Test Method Results

One advantage of the absorbent structures of the present disclosure is the superior superabsorbent particle capture and retention, particularly at low adhesive add-on levels. In a first test, Example 1, Example 2, Example 3, and Comparative Example 1 test methods were tested according to the Free SAM Determination Test Method as a measure of the superabsorbent particle stabilization within each of the Example 1, Example 2, Example 3, and Comparative Example 1 structures. As shown below in TABLE 1, the Example 1 and Example 2 structures performed well, even at low adhesive add-on levels. Comparatively, the Comparative Example 1, surprisingly, performed significantly worse than the Example 1 and Example 2 structures even at a substantially higher adhesive add-on rate than the Example 1 and Example 2 structures.

TABLE 1

| Sample | Free SAM | |
|---|---|---|
| Example 1 | 0.3 | g |
| (2.85% adhesive add-on) | | |
| Example 2 | 0.16 | g |
| (3.56% adhesive add-on) | | |
| Example 3 | 3.52 | g |
| (1.44% adhesive add-on) | | |
| Comparative Example 1 | 1.1 | g |
| (5% adhesive add-on) | | |

### SAM Penetration Test Method Results

As evidenced above by the Free SAM Determined Test Method results, one advantage of the absorbent structures of the present disclosure is that the superabsorbent particles are captured and immobilized to a greater extent that in prior art process and structures. A few benefits of this result is that more superabsorbent particles are captured with the adhesive (and at a lower add-on rate than prior art structures) such that less particles are filtered through and captured within the reinforcing web materials 329. To demonstrate this feature of the absorbent structures of the present disclosure, the Example 1 and Comparative Example 1 structures were tested according to the SAM Penetration Test Method, which is a measure of capture of the superabsorbent particles of the structures within the reinforcing web material. The test method results returned a SAM penetration value of approximately 64% for Example 1 structures compared to a SAM penetration value of approximately 72% for the Comparative Example 1 structures.

Were it not for the Free SAM Determination Test Method Such a result is somewhat surprising. For instance, the Example 1 structures utilize significantly less adhesive than the Comparative Example 1 structures yet are better able to retain the superabsorbent particles as part of the adhesive matrix and prevent the superabsorbent particles from penetrating into the reinforcing web material.

### Cradle Intake Test Method Results

One result of the lower penetration of the superabsorbent particles of the Example 1 structures of the present disclosure is that it is believed this feature results in superior intake performance. For example, less adhesive is utilized in the Example 1 structures as compared to the Comparative Example 1 structures, which represents less blocking of the liquid as it penetrates into the structures by the hydrophobic adhesive. Additionally, as subsequent insults are applied to the absorbent structures, it is believed that the lower amount of superabsorbent particle penetration into the reinforcing web material results in a continued ability of the absorbent structure to intake liquid at a fast rate. It is believed that the relative absence of superabsorbent particles within the reinforcing web material results in an ability of the absorbent structure to maintain void volume space within the structure, representing a place for liquid to gather. Accordingly, where the reinforcing web material includes a greater amount of penetrated superabsorbent particles, we would expect the intake rate of such absorbent structure to decrease as the available void volume of the structure decreases due to the swollen superabsorbent particles blocking off portions of the reinforcing web material. As seen in the Cradle Intake Test Method results shown below in TABLE 2, this belief appears to hold true, as the Example 1 structures perform superior with respect to a third cradle intake time.

TABLE 2

| | 1^{st} Cradle Intake | 2^{nd} Cradle Intake | 3^{rd} Cradle Intake |
|---|---|---|---|
| Sample | Time (s) | Time (s) | Time (s) |
| Example 1 | 20 | 13 | 20 |
| Comparative | 20 | 12 | 30 |
| Example 1 | | | |

### Free SAM Determination Test Method

To perform the Free SAM Determination Test Method, a sample formed product or absorbent core is utilized. If a sample formed product is utilized, it is deconstructed leaving just the absorbent core. The deconstruction may be performed by simply tearing layers away from the absorbent core. In some instances, a commercially available adhesive de-activator may be used, such as freeze-spray type adhesive remover/deactivator may be utilized to help separate the layers.

Once the absorbent core has been isolated, the core layers may be peeled apart by grasping, at one end of the core, a first of the outside material layers in a first hand and the rest of the core (e.g. the other outer material layer and the inner reinforcing material) in a second hand. The first outside layer is then peeled away utilizing a peeling motion with the first hand. The peeling is performed over a capture box which captures any free superabsorbent particles which fall out during the peeling motion.

Next, the separated first outer layer is given two slight shakes over the capture box to capture any superabsorbent particles falling away from the first outer layer. The rest of the remaining absorbent core is given two slight shakes over the capture box as well. The weight of the superabsorbent particles in the capture box may be recorded at this time.

The second of the outside material layers is then grasped in the first hand and the remaining core (e.g. the inner reinforcing layer) is grasped the second hand. The second outside material layer is then peeled away from the inner reinforcing layer, with the peeling performed over the capture box to capture any superabsorbent particles which fall out during the peeling motion.

Next, the second outer layer is given two slight shakes over the capture box to capture any superabsorbent particles falling away from the second outer layer. The remaining inner reinforcing material is given two slight shakes over the capture box as well. The weight of the total superabsorbent particles in the capture box is recorded at this time.

Typically, five samples may be measured according to this test method. The output result of the Free SAM Determination Test Method may then be the average of the total weighed superabsorbent particles captured within the capture box of the five samples.

### SAM Penetration Test Method

To perform the SAM Penetration Test Method, a sample formed product or absorbent core is utilized. If a sample formed product is utilized, it is deconstructed leaving just the absorbent core. The deconstruction may be performed by simply tearing layers away from the absorbent core. In some instances, a commercially available adhesive de-activator may be used, such as freeze-spray type adhesive remover/deactivator may be utilized to help separate the layers.

Once the absorbent core has been isolated, the core layers may be carefully separated by grasping, at one end of the core, a first of the outside material layers in a first hand and the rest of the core (e.g. the other outer material layer and the inner reinforcing material) in a second hand. The first outside layer is then slowly peeled away utilizing a peeling motion with the first hand. The peeled first outside material layer is weighed.

Next, the second of the outside material layers is grasped in the first hand and the rest of the core (e.g. the inner reinforcing material) is grasped in the second hand. The second outside layer is then slowly peeled away utilizing a peeling motion with the first hand. The peeled second outside material layer is weighed. The remaining inner reinforcing material is then weighed.

The dimensions of the inner reinforcing material are known and, along with a basis weight of the material, may be used to calculate a weight of the inner reinforcing material. For example, if the inner reinforcing material is 325 mm long by 115 mm wide, and has a basis weight of 40 gsm, the weight of the inner reinforcing material may be calculated as to be 1.495g (e.g. 0.325×0.115×40=1.495g). Next, a combination weight of the inner reinforcing material and the applied adhesive may be calculated according to the applied add-on rate of the adhesive. For example, if the adhesive is applied over the entire width and length of the inner reinforcing material, and applied at an add-on rate of 11.5 gsm, the adhesive weight may be calculated to be 0.43g. Accordingly, a combined inner reinforcing material and adhesive weight is then 1.495+0.425=1.92g. From this combined inner reinforcing material and adhesive weight, an amount of superabsorbent particles contained within or on the inner reinforcing material can be calculated by subtracting the combined inner reinforcing material and adhesive weight from the measured weight of the inner reinforcing material. For example, where the inner reinforcing material was weighed to be 6.88 g, the amount of superabsorbent particles retained on or within the inner reinforcing material is 6.88g−1.92g=4.96g. In some cases, it may be useful to express this value as a percentage of the measured weight of inner reinforcing material by diving the calculated amount of superabsorbent particles by the measured weight of inner reinforcing material and multiplying by 100. This value is called the SAM penetration value herein. For the illustrated example above, the SAM penetration value would be 4.96g/6.88 g×100=72%. Typically, ten products are measured in this manner and an average of the resulting values are used when reporting values.

### Cradle Intake Test Method

The Cradle Intake Test Method is performed on whole products and is typically performed on ten samples of each code, with the resulting measurements averaged to report final values. The Cradle Intake Test Method utilizes a test cradle apparatus simulating the body curvature of a child. The apparatus consists of a plexiglass box with internal plexiglass walls angled towards a transverse centerline of the box. The angle of the internal walls is approximately 45 degrees, with the internal walls meeting at the transverse centerline of the box and forming rounded connection have a radius of curvature of approximately 3.8 cm.

For each single sample, the sample is opened to a flat (side seams are severed if relevant) and stretched-out configuration and a center of the product is marked-utilizing a ruler or other measuring device to find the center. Next, and insult point is marked. For a step 3 diaper or diaper pant, the insult point is marked as 7.5 cm forward (toward a front of the article) from the marked center.

Prior to being placed within the cradle, the sample product is weighed to the nearest 0.1g using a suitable measurement device—for example an electro-balance readable to 0.01 g. If the product includes containment flaps, the flaps should be slightly lifted from the liner by running a finger underneath the containment flaps to ensure that no portion of the flaps are adhered to the liner.

With the product in the cradle, an end of a clear tubing-such as Masterflex clear tubing L/S 16, having an exit diameter of approximately 1 cm—is positioned approximately 1 cm away from the marked target insult location, with the tubing end pointing directly at the target insult location.

The clear tubing is connected to a suitable pump apparatus that is capable of controlling a flow rate (for example, a Cole-Parmer peristaltic pump P/N 07551-20, with pump head P/N 77201-60). The pump is connected to a reservoir containing a saline solution (0.9±0.0005% (w/w) aqueous isotonic saline) heated to 37±1 degree C.

For a step 3 size product, the total volume per insult is 60 ml, and the fluid is delivered at a rate of 15 ml/s. Accordingly, the pump is set to appropriately control to these parameters. Utilizing a stopwatch, a first insult is timed, beginning when fluid contacts the sample. The timer is stopped once all of the insulted liquid has penetrated below the surface of the sample, and the time is recorded to the nearest 0.01 seconds. Failures can occur whereby more than a few drops of liquid migrate to non-absorptive portions of the sample, where the liquid runs into the cradle (for example, by leaking from the sample), and whereby fluid pools for longer than 5 minutes. These failures should be noted and the data not included in the reported results.

After recording the time, a countdown timer set for 15 minutes is initiated. Once the 15 minute period is done, the above process is repeated for subsequent insults. The recorded times may be reported as 1st, 2nd, 3rd, etc. Cradle Intake Times.

### Adhesive Distribution Test Method

Samples to be imaged are first stained with osmium tetroxide fume so that the adhesive selectively absorbs the osmium in sufficient quantities to allow it to be more easily contrasted from the super absorbent and polymeric fibers during Micro-CT imaging. A sample is stained by placing it in a closable, air-tight chamber to which a small vial of osmium tetroxide is added. The chamber is then immediately sealed and the osmium tetroxide is allowed to interact with the sample for at least 24 hours. Since osmium tetroxide is highly toxic, the staining procedure is carried out in a fume hood. After the 24 hour period, the adhesive should become blackened in appearance. After re-opening the chamber, it is allowed to air out in the hood for another 24 hours to ensure any unreacted osmium tetroxide is allowed to harmlessly escape. After the second 24 hour period, the sample is now ready to be imaged in the Micro-CT,

A Bruker SkyScan Model 1272 Micro-CT, or equivalent, is used to image a portion of the stained sample. Example X-ray scanning conditions include the following:
Voltage (KV)=35
Current (uA)=231
Image Pixel Size (um)=8.0
Rotation Step (deg)=0.20
Frame Averaging=5
   The sample piece must be oriented so that machine-direction length is held in the vertical position during the scanning process. After initial x-ray scanning, the rotational x-ray images are then reconstructed using Bruker's NRecon software, or equivalent on a different vendor's system. The gray-scale reconstructed image slices are used for the adhesive distribution analysis.

The image analysis software platform used to perform the adhesive distribution measurements may be a QWIN Pro (Version 3.2.1) available from Leica Microsystems, having an office in Heerbrugg, Switzerland. The custom-written image analysis algorithm ‘Z-Adhesive Distribution’ was used to process and perform measurements of grayscale Micro-CT images using Quantimet User Interactive Programming System (QUIPS) language. The custom image analysis algorithm shown below was performed directly on the gray-scale reconstructed image slices that were stored on a storage device. The custom image analysis algorithm is shown below.
NAME: Z-Adhesive Distribution
PURPOSE: Measures z-distribution of osmium stained adhesive on ActivTech/Blizzard Substrates
CONDITIONS: Images acquired on the Bruker SkyScan 1272 Micro-CT
DATE: Aug. 12, 2020
AUTHOR: D. G. Biggs
SET-UP
Clear Accepts
DATA FILES OPENED
Open File (C: \Data\102888-Graverson\totdistribution.xls, channel #2)
Open File (C: \Data\102888-Graversonladhesivedistribution.xls, channel #1)
Configure (Image Store 1968×504, Grey Images 201, Binaries 32)
Calvalue=8.00 um/px
CALVALUE=8.00
Calibrate (CALVALUE CALUNITS$ per pixel)
Measure frame (x 160, y 2, Width 1600, Height 502)
Image frame (x 0, y 0, Width 1968, Height 504)
Enter Results Header
File Results Header (channel #1)
File Line (channel #1)
File Results Header (channel #2)
File Line (channel #2)
PauseText (“Enter sample image file prefix name.”)
Input (TITLE$)
File (TITLE$, channel #1)
File Line (channel #1)
For (IMAGE=100 to 900, step 100)
Clear Feature Histogram #1
Clear Feature Histogram #3
DEFINE BINARY GRAPHICS VARIABLES
GRAPHORGX=250
IMAGE ACQUISITION AND DETECTION
ACQOUTPUT=0
Location of Micro-CT images to be analyzed
ACQFILE$= “C: \Images\102888-Graverson\Code 2-Blizzard Tech
Osmium\”+TITLE$+″ “+STR$ (IMAGE)+”.JPG″
Read image (from file ACQFILE$ into ACQOUTPUT)
Colour Transform (Mono Mode)
Detect all material
Detect (whiter than 33, from Image0 into Binary0)
IMAGE PROCESSING
PauseText (“Accept the primary structure and exclude any outlying debris.”)
Binary Edit [PAUSE] (Accept from Binary0 to Binary1, nib Fill, width 2)
Binary Amend (Open from Binary1 to Binary1, cycles 1, operator Disc, edge erode on)
Binary Amend (Close from Binary1 to Binary2, cycles 120, operator Disc, edge erode on)
Binary Identify (FillHoles from Binary2 to Binary3)
Binary Amend (Open from Binary3 to Binary4, cycles 5, operator Disc, edge erode on)
BOLEAN AND MEASUREMENT
For (BINGRAPH=1 to 26, step 1)
GRAPHORGY=2
GRAPHNX=1
GRAPHNY=1
GRAPHWID=50
GRAPHHGHT=502
GRAPHTHIK=1
GRAPHORNT=0
GRAPHOUT=13
Graphics (Inverted Grid, GRAPHNX×GRAPHNY Lines, Grid Size GRAPHWID×GRAPHHGHT, Origin GRAPHORGX×GRAPHORGY,
Thickness GRAPHTHIK, Orientation GRAPHORNT, to GRAPHOUT Cleared)
Binary Logical (C=A AND B: C Binary5, A Binary4, B Binary13)
CENTER YPOS
Measure feature (plane Binary5, 32 ferets, minimum area: 10, grey image: Colour0) Selected parameters: UserDef1, YCentroid
Feature Expression (UserDef1 (all features), title CalcA=(PYCENTROID (FTR)-252))
GREYUTILIN=0
GREYUTILOUT=1
Shift Grey Image
If (PUSERDEF1 (FTR)<0)
DISTANCE=(PUSERDEF1 (FTR)**2)**0.5
SHIFT.SIZE=DISTANCE
PCT/CN2021/125981 WO 2023/070240
SHIFT.DIRN=270
Grey Util (Shift GREYUTILIN to GREYUTILOUT by SHIFT.SIZE at SHIFT.DIRN degs)
Endif
If (PUSERDEF1 (FTR)>0)
DISTANCE=PUSERDEF1 (FTR)
SHIFT.SIZE=DISTANCE
SHIFT.DIRN=90
Grey Util (Shift GREYUTILIN to GREYUTILOUT by SHIFT.SIZE at SHIFT.DIRN degs)
Endif
If (PUSERDEF1 (FTR)=0)
Grey Util (Copy Image0 to Image1)
Endif
Display (Image0(on), frames (on,on), planes (off,off,off,off,off,off), lut 0, x 0, y 0, z 1, Reduction off)
DETECT AFTER CENTERING
Detect adhesive
Detect (whiter than 84, from Image1 into Binary10)
Binary Amend (Close from Binary10 to Binary10, cycles 1, operator Disc, edge erode on)
Binary Amend (Open from Binary10 to Binary11, cycles 1, operator Disc, edge erode on)
Detect all material
Detect (whiter than 33, from Image1 into Binary0)
Binary Amend (Close from Binary0 to Binary0, cycles 1, operator Disc, edge erode on)
Binary Amend (Open from Binary0 to Binary0, cycles 1, operator Disc, edge erode on)
MEASURE ADHESIVE Z-DISTRIBUTION
GRAPHORGY=2
GRAPHNX=1
GRAPHNY=1
GRAPHWID=50
GRAPHHGHT=502
GRAPHTHIK=1
GRAPHORNT=0
GRAPHOUT=12
Graphics (Inverted Grid, GRAPHNX×GRAPHNY Lines, Grid Size GRAPHWID×GRAPHHGHT, Origin GRAPHORGX×GRAPHORGY,
Thickness GRAPHTHIK, Orientation GRAPHORNT, to GRAPHOUT Cleared)
Binary Logical (C=A AND B: C Binary6, A Binary12, B Binary11)
Measure feature (plane Binary6, 32 ferets, minimum area: 10, grey image: Image1)
Selected parameters: Area, UserDef2, YCentroid
Feature Expression (UserDef2 (all features), title YFEAT=PYCENTROID (FTR)*CALVALUE)
Feature Histogram #1 (Y Param Area, X Param UserDef2, from 0, to 4032., linear, 40 bins)
Feature Histogram #2 (Y Param Area, X Param UserDef2, from 0, to 4032., linear, 40 bins)
MEASURE TOTAL MATERIAL Z-DISTRIBUTION
Binary Logical (C=A AND B: C Binary7, A Binary12, B Binary0)
Measure feature (plane Binary7, 32 ferets, minimum area: 10, grey image: Image1)
Selected parameters: Area, X FCP, Y FCP, UserDef2, YCentroid
Feature Expression (UserDef2 (all features), title YFEAT=PYCENTROID (FTR)*CALVALUE)
Feature Histogram #3 (Y Param Area, X Param UserDef2, from 0, to 4032., linear, 40 bins)
Feature Histogram #4 (Y Param Area, X Param UserDef2, from 0, to 4032., linear, 40 bins)
GRAPHORGX=GRAPHORGX+50
Next (BINGRAPH)
Display Feature Histogram Results (#2, horizontal, differential, bins+graph (Y axis linear), statistics)
Data Window (10, 871, 640, 300)
Display Feature Histogram Results (#4, horizontal, differential, bins+graph (Y axis linear), statistics)
Data Window (962, 880, 640, 300)
FILE ADHESIVE AND MATERIAL HISTOGRAMS FOR CURRENT IMAGE
File Feature Histogram Results (#1, differential, statistics, bin details, channel #1)
File Line (channel #1)
File Feature Histogram Results (#3, differential, statistics, bin details, channel #2)
File Line (channel #2)
File Line (channel #2)
MEASURE MEAN SUBSTRATE THICKNESS
MFLDIMAGE=4
Measure field (plane MFLDIMAGE, into FLDRESULTS (1), statistics into FLDSTATS (7,1))
Selected parameters: Area
MEANTHICK=FLDRESULTS (1)/(CALVALUE*1330)
File (“Mean Substrate Thickness (um)= “, channel #1)
File (MEANTHICK, channel #1, 2 digits after ‘.’)
File Line (channel #1)
File Line (channel #1)
Next (IMAGE) 61
FILE CUMMULATIVE ADHESIVE AND MATERIAL HISTOGRAMS FOR CURRENT SLIDE
File Feature Histogram Results (#2, differential, statistics, bin details, channel #1)
File Feature Histogram Results (#4, differential, statistics, bin details, channel #2)
CLOSE DATA FILES
Close File (channel #1)
Close File (channel #2)
END

The adhesive distribution in the z-direction data are exported directly to an EXCEL® spreadsheet. Individual adhesive and total material z-distribution histograms are exported for data acquired from each of the analyzed slices of the micro-CT image as well as a cumulative histogram for data from all nine slices. These latter cumulative histograms were used for calculating the percentage of adhesive in each one-third layer of the thickness of the micro-CT image for a single slice. The area units are shown in the histogram are in square microns. In order to determine the histogram location of the top and bottom surface boundaries of the material, a 95 percent of total area rule was used on the total material histogram. In other words, when approaching the top and bottom material edges of the histogram, the surface boundary was considered to be the first histogram bin when a minimum of 2.5 percent material area had been encountered. These bin boundaries were then transposed over to the adhesive only cumulative histogram to determine the percentages of adhesive area present in the top, middle and bottom one-third histogram bins, inclusive of the calculated boundary bins. In cases where the number of bins was not evenly divisible by three (e.g. 8, 10, 14, etc.), a rotation technique was used to calculate adhesive percentages in each one-third layer of the material. For example, in the first encounter of a fourteen bin thickness, the top layer was four bins, the middle five, and the bottom five. During the next encounter, the top layer was five bins, the middle four, and the bottom five. If a third encounter occurs, the bottom layer would have one less or one more bin than the top and middle. If a fourth encounter occurs, the top layer again becomes the one containing one less or one more bin than the other two layers. This rotating method continues as required by the data.

The final sample mean adhesive percentage values for each one-third layer of z-distribution depth is based on an N=7 analysis from seven, separate, subsample regions each possessing four adjacently cut cross-sections. A comparison between different samples can be performed using a Student's T analysis at the 90 percent confidence level.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EMBODIMENTS

In a first embodiment, an absorbent article may extend in a longitudinal and a lateral direction and comprise an inner bodyside liner; an outer cover; and an absorbent structure disposed between the inner bodyside liner and the outer cover, with the absorbent structure comprising a top facing material layer disposed adjacent the bodyside liner; a bottom facing material layer disposed adjacent the outer cover; a lofty nonwoven reinforcing material disposed between the top facing material layer and the bottom facing material layer; and a first absorbent layer comprising superabsorbent particles intermixed with adhesive filaments disposed between the top facing material layer and the lofty nonwoven reinforcing material or between the lofty nonwoven reinforcing material and the bottom facing material layer, wherein the superabsorbent particles may be present in an amount greater than or equal to 90%, by weight of absorbent material between the top facing material layer and the bottom facing material layer, and the adhesive filaments may form a three-dimensional mesh network comprising network adhesive filaments with the superabsorbent particles immobilized within the mesh network, the network adhesive filaments and superabsorbent particles extending throughout a three-dimensional space defined by the network adhesive filaments and the superabsorbent particles, with the network adhesive filaments extending in random orientations throughout the three-dimensional space.

In a second embodiment, the absorbent article of the first embodiment may further comprise only the first absorbent layer disposed between the top facing material layer and the lofty nonwoven reinforcing material or between the lofty nonwoven reinforcing material and the bottom facing material layer and may not comprise a second absorbent layer disposed between the other of the top facing material layer and the lofty nonwoven reinforcing material or between the lofty nonwoven reinforcing material and the bottom facing material layer.

In a third embodiment, the absorbent structure of the absorbent article of the second embodiment may comprise superabsorbent particles which are disposed within the absorbent structure at an average basis weight of between 50 gsm and 600 gsm.

In a fourth embodiment, the absorbent structure of the absorbent article of any of the first through third embodiments may be configured wherein the first absorbent layer is disposed between the top facing material layer and the lofty nonwoven reinforcing material, and the absorbent structure may further comprise a second absorbent layer disposed between the lofty nonwoven reinforcing material and the bottom facing material layer.

In a fifth embodiment, the absorbent structure of the absorbent article of the fourth embodiment may comprise superabsorbent particles within the first absorbent layer at an amount between 100 gsm and 250 gsm, and superabsorbent particles within the second absorbent layer at an amount between 100 gsm and 250 gsm.

In a sixth embodiment, the absorbent structure of the absorbent article of the fifth embodiment may comprise superabsorbent particles within the first absorbent layer present in an amount measured in gsm that is greater than or equal to 75% of an amount of the superabsorbent particles within the second absorbent layer, also as measured in gsm.

In a seventh embodiment, the absorbent structure of the absorbent article of any of the fourth through sixth embodiments may comprise adhesive filaments which are present in an amount less than 5%, by weight of the superabsorbent particles.

In an eighth embodiment, the absorbent structure of the absorbent article of any of the fourth through sixth embodiments may comprise adhesive filaments which are present in an amount less than 4%, by weight of the superabsorbent particles.

In a ninth embodiment, the absorbent structure of the absorbent article of any of the third through eighth embodiments may comprise adhesive filaments and superabsorbent particles which are intermixed prior to deposition onto a forming surface to form the absorbent structure, and may lack adhesive other than the adhesive intermixed with the superabsorbent particles.

In a tenth embodiment, the absorbent article of any of the first through ninth embodiments may lack a surge or distribution layer between the absorbent structure and the bodyside liner.

In an eleventh embodiment, the absorbent structure of the absorbent article of any of the first through tenth embodiments may be cellulose free.

In a twelfth embodiment, an absorbent article may extend in a longitudinal and a lateral direction and comprise: an inner bodyside liner; an outer cover; and an absorbent structure disposed between the inner bodyside liner and the outer cover, wherein the absorbent structure may comprise a top facing material layer disposed adjacent the bodyside liner, a bottom facing material layer disposed adjacent the outer cover, a lofty nonwoven reinforcing material disposed between the top facing material layer and the bottom facing material layer, a first absorbent layer comprising superabsorbent particles intermixed with adhesive filaments disposed between the top facing material layer and the lofty nonwoven reinforcing material, the superabsorbent particles of the first absorbent layer being present in an amount between 100 gsm and 250 gsm, and a second absorbent layer comprising superabsorbent particles intermixed with adhesive filaments disposed between the lofty nonwoven reinforcing material the bottom facing material layer, the superabsorbent particles of the second absorbent layer being present in an amount between 100 gsm and 250 gsm, with the superabsorbent particles being present in an amount greater than or equal to 90%, by weight of absorbent material between the top facing material layer and the bottom facing material layer, and a total quantity of adhesive disposed between the top facing material layer and the bottom facing material layer may be present in an amount less than 4.5%, by weight of the superabsorbent particles present between the top facing material layer and the bottom facing material layer, and where the adhesive filaments may form a three-dimensional mesh network comprising network adhesive filaments with the superabsorbent particles immobilized within the mesh network, the network adhesive filaments and superabsorbent particles extending throughout a three-dimensional space defined by the network adhesive filaments and the superabsorbent particles, with the network adhesive filaments extending in random orientations throughout the three-dimensional space.

In a thirteenth embodiment, the absorbent article of the twelfth embodiment may comprise superabsorbent particles of the first absorbent layer which are present in an amount greater than 0 gsm and less than 200 gsm, and where the superabsorbent particles of the second absorbent layer are present in an amount greater than 0 gsm and less than 200 gsm.

In a fourteenth embodiment, the absorbent article of any of the twelfth and thirteenth embodiments, may further comprise a total quantity of adhesive disposed between the top facing material layer and the bottom facing material layer being present in an amount less than 3.0%, by weight of the superabsorbent particles present between the top facing material layer and the bottom facing material layer, and the absorbent structure has a SAM penetration value of less than or equal to 65%, according to the SAM Penetration Test Method.

In a fifteenth embodiment, the absorbent article of any of the twelfth through fourteenth embodiments may further comprise a total quantity of adhesive disposed between the top facing material layer and the bottom facing material layer being present in an amount less than 3.0%, by weight of the superabsorbent particles present between the top facing material layer and the bottom facing material layer, and a total amount of free superabsorbent particles within the absorbent structure being less than 0.5g, according to the Free SAM Determination Test Method.

In a sixteenth embodiment, the absorbent structure of the absorbent article of any of the twelfth through fifteenth embodiments may be cellulose free.

In a seventeenth embodiment, the absorbent article of any of the twelfth through sixteenth embodiments may further comprise the adhesive filaments and the superabsorbent particles intermixing prior to deposition onto a forming surface to form the absorbent structure, with the absorbent structure lacking adhesive other than the adhesive intermixed with the superabsorbent particles.

In an eighteenth embodiment, an absorbent article may extend in a longitudinal and a lateral direction and comprise an inner bodyside liner; an outer cover; and an absorbent structure disposed between the inner bodyside liner and the outer cover comprising superabsorbent particles present in an amount greater than or equal to 90%, by weight of absorbent material of the absorbent structure, the absorbent structure further comprising a top facing material layer disposed adjacent the bodyside liner; a bottom facing material layer disposed adjacent the outer cover; a lofty nonwoven reinforcing material disposed between the top facing material layer and the bottom facing material layer; a first absorbent layer comprising superabsorbent particles intermixed with adhesive filaments disposed between the top facing material layer and the lofty nonwoven reinforcing material, the superabsorbent particles of the first absorbent layer being present in an amount greater than 0 gsm and less than 200 gsm; and a second absorbent layer comprising superabsorbent particles intermixed with adhesive filaments disposed between the lofty nonwoven reinforcing material the bottom facing material layer, the superabsorbent particles of the second absorbent layer being present in an amount greater than 0 gsm and less than 200 gsm, with a total quantity of adhesive disposed between the top facing material layer and the bottom facing material layer being present in an amount less than 4.0%, by weight of the superabsorbent particles present between the top facing material layer and the bottom facing material layer, wherein the adhesive filaments may form a three-dimensional mesh network comprising network adhesive filaments with the superabsorbent particles immobilized within the mesh network, the network adhesive filaments and superabsorbent particles extending throughout a three-dimensional space defined by the network adhesive filaments and the superabsorbent particles, with the network adhesive filaments extending in random orientations throughout the three-dimensional space, and wherein the absorbent structure has a SAM penetration value of the absorbent structure is less than or equal to 65%, according to the SAM Penetration Test Method.

In a nineteenth embodiment, the absorbent article of the eighteenth embodiment may further comprise a total quantity of adhesive disposed between the top facing material layer and the bottom facing material layer being present in an amount less than 3.0%, by weight of the superabsorbent particles present between the top facing material layer and the bottom facing material layer.

In a twentieth embodiment, the absorbent article of any of the eighteenth and nineteenth embodiments may further comprise wherein a total amount of free superabsorbent particles within the absorbent structure is less than 0.5g, according to the Free SAM Determination Test Method.

In a twenty-first embodiment, the absorbent structure of the absorbent article of any of the eighteenth through twentieth embodiments may be cellulose free.

In a twenty-second embodiment, the absorbent article of any of the eighteenth through twenty-first embodiments may further comprise the adhesive filaments and the superabsorbent particles intermixing prior to deposition onto a forming surface to form the absorbent structure, with the absorbent structure lacking adhesive other than the adhesive intermixed with the superabsorbent particles.

In a twenty-third embodiment, a method of forming an absorbent structure may comprise feeding a first stream of superabsorbent particles toward a first substrate material layer moving in a machine direction, the first stream of superabsorbent particles having a first side and a second side; spraying, with a first adhesive applicator having a first adhesive nozzle, the first side of the first stream of superabsorbent particles with a first adhesive, the first adhesive contacting the first stream of superabsorbent particles and intermixing with the superabsorbent particles of the first stream of superabsorbent particles prior to said superabsorbent particles depositing onto the first substrate material layer, the first adhesive contacting the first stream of superabsorbent particles at a first contact point having a first height as measured from the first substrate material layer; spraying, with a second adhesive applicator having a second adhesive nozzle, the second side of the first stream of superabsorbent particles with a second adhesive, the second adhesive contacting the first stream of superabsorbent particles and intermixing with the superabsorbent particles of the first stream of superabsorbent particles prior to said superabsorbent particles depositing onto the first substrate material layer, the second adhesive contacting the first stream of superabsorbent particles at a second contact point having a second height as measured from the first substrate material layer, the first height being different from the second height; depositing the intermixed superabsorbent particles of the first stream of superabsorbent particles, first adhesive, and second adhesive onto the first substrate material layer; covering the mixture of the superabsorbent particles of the first stream of superabsorbent particles, the first adhesive, and the second adhesive with a lofty nonwoven reinforcing material; feeding a second stream of superabsorbent particles toward the lofty nonwoven reinforcing material, the second stream of superabsorbent particles having a first side and a second side; spraying, with a third adhesive applicator having a third adhesive nozzle, the first side of the second stream of superabsorbent particles with a third adhesive, the third adhesive contacting the second stream of superabsorbent particles and intermixing with the superabsorbent particles of the second stream of superabsorbent particles prior to said superabsorbent particles depositing onto the lofty nonwoven reinforcing material; spraying, with fourth first adhesive applicator having a second adhesive nozzle, the second side of the second stream of superabsorbent particles with a fourth adhesive, the fourth adhesive contacting the second stream of superabsorbent particles and intermixing with the superabsorbent particles of the second stream of superabsorbent particles prior to said superabsorbent particles depositing onto the lofty nonwoven reinforcing material; depositing the intermixed superabsorbent particles of the second stream of superabsorbent particles, third adhesive, and fourth adhesive onto the lofty nonwoven reinforcing material; and covering the mixture of the superabsorbent particles of the second stream of superabsorbent particles, the third adhesive, and the fourth adhesive with a second substrate material layer to form the absorbent structure.

In a twenty-fourth embodiment, the method of the twenty-third embodiment may further comprise wherein the first side of the first stream of superabsorbent particles is upstream of the second side of the first stream of superabsorbent particles in the machine direction and wherein the first contact point is closer to the first substrate material layer than the second contact point.

In twenty-fifth embodiment, the method of the twenty-third embodiment may further comprise wherein the first side of the first stream of superabsorbent particles is upstream of the second side of the first stream of superabsorbent particles in the machine direction and wherein the first contact point is further away from the first substrate material layer than the second contact point.

In a twenty-sixth embodiment, the method of any of the twenty-third through twenty-fifth embodiments may further comprise wherein the first height is located between 4 mm and 40 mm from the first substrate material layer.

In a twenty-seventh embodiment, the method of any of the twenty-third through twenty-sixth embodiments may further comprise wherein the first height is spaced from the second height by between 3 mm and 9.5 mm.

In a twenty-eighth embodiment, the method of the twenty-seventh embodiment may further comprise wherein the second adhesive applicator is oriented at a second angle of between 45 degrees and 75 degrees with respect to the machine direction, and wherein the first angle is the same as the second angle.

In a twenty-ninth embodiment, the method of any of the twenty-third through twenty-eighth embodiments may further comprise severing first layer of material, the mixture of the superabsorbent particles of the first stream of superabsorbent particles, the first adhesive, and the second adhesive, and the second substrate material layer into individual absorbent bodies.

In a thirtieth embodiment, the method of any of the twenty-third through twenty-ninth embodiments may further comprise wherein the method does not comprise any additional adhesive application steps which apply adhesive between the first material layer and the second material layer.

In a thirty-first embodiment, the method of any of the twenty-third through thirtieth embodiments may further comprise wherein the formed absorbent structure has a SAM penetration value of the absorbent structure is less than or equal to 65%, according to the SAM Penetration Test Method.

In a thirty-second embodiment, the method of any of the twenty-third through thirty-first embodiments may further comprise wherein a total amount of free superabsorbent particles within the absorbent structure is less than 0.5g, according to the Free SAM Determination Test Method.

In a thirty-third embodiment, a method of forming an absorbent structure may comprise feeding a first stream of superabsorbent particles toward a first substrate material layer, the first stream of superabsorbent particles having a first side and a second side, wherein the first stream of superabsorbent particles is fed such that the superabsorbent particles of the first stream of superabsorbent particles produces a first layer of superabsorbent particles having a basis weight of superabsorbent particles of greater than 0 gsm and less than 200 gsm; spraying, with a first adhesive applicator having a first adhesive nozzle, the first side of the first stream of superabsorbent particles with a first adhesive, the first adhesive contacting the first stream of superabsorbent particles and intermixing with the superabsorbent particles of the first stream of superabsorbent particles prior to said superabsorbent particles depositing onto the first substrate material layer; spraying, with a second first adhesive applicator having a second adhesive nozzle, the second side of the first stream of superabsorbent particles with a second adhesive, the second adhesive contacting the first stream of superabsorbent particles and intermixing with the superabsorbent particles of the first stream of superabsorbent particles prior to said superabsorbent particles depositing onto the first substrate material layer; depositing the intermixed superabsorbent particles of the first stream of superabsorbent particles, first adhesive, and second adhesive onto the first substrate material layer, wherein the total amount of adhesive intermixed with the superabsorbent particles of the first layer of superabsorbent particles is less than 4%, by weight, of the weight of the superabsorbent particles within the first layer of superabsorbent particles; covering the mixture of the superabsorbent particles of the first stream of superabsorbent particles, the first adhesive, and the second adhesive with a lofty nonwoven reinforcing material; feeding a second stream of superabsorbent particles toward the lofty nonwoven reinforcing material, the second stream of superabsorbent particles having a first side and a second side, wherein the second stream of superabsorbent particles is fed such that the superabsorbent particles of the second stream of superabsorbent particles produces a second layer of superabsorbent particles having a basis weight of superabsorbent particles of greater than 0 gsm and less than 200 gsm; spraying, with a third adhesive applicator having a third adhesive nozzle, one of the first side and the second side of the second stream of superabsorbent particles with a third adhesive, the third adhesive contacting the second stream of superabsorbent particles and intermixing with the superabsorbent particles of the second stream of superabsorbent particles prior to said superabsorbent particles depositing onto the lofty nonwoven reinforcing material; spraying, with a fourth first adhesive applicator having a fourth adhesive nozzle, the other of the first side and the second side of the second stream of superabsorbent particles with a fourth adhesive, the fourth adhesive contacting the second stream of superabsorbent particles and intermixing with the superabsorbent particles of the second stream of superabsorbent particles prior to said superabsorbent particles depositing onto the lofty nonwoven reinforcing material; depositing the intermixed superabsorbent particles of the second stream of superabsorbent particles, third adhesive, and fourth adhesive onto the lofty nonwoven reinforcing material, and covering the mixture of the superabsorbent particles of the second stream of superabsorbent particles, the third adhesive, and the fourth adhesive with a second substrate material layer to form the absorbent structure, wherein the total amount of adhesive intermixed with the superabsorbent particles of the second layer of superabsorbent particles is less than 4%, by weight, of the weight of the superabsorbent particles within the second layer of superabsorbent particles.

In a thirty-fourth embodiment, the method of the thirty-third embodiment may further comprise wherein the first adhesive contacts the first stream of superabsorbent particles at a first contact point having a first height as measured from the first substrate material layer, the third adhesive contacts the first stream of superabsorbent particles at a third contact point having a third height as measured from the first substrate material layer, the second adhesive contacts the second stream of superabsorbent particles at a second contact point having a second height as measured from the first substrate material layer, the fourth adhesive contacts the second stream of superabsorbent particles at a fourth contact point having a fourth height as measured from the first substrate material layer, and the first height is different from the third height and the second height is different from the fourth height.

In a thirty-fifth embodiment, the method of any of the thirty-third through thirty-fourth embodiments may further comprise wherein the method does not comprise any additional adhesive application steps which apply adhesive between the first material layer and the second material layer.

In a thirty-sixth embodiment, the method of any of the thirty-third through thirty-fifth embodiments may further comprise wherein the formed absorbent structure has a SAM penetration value of the absorbent structure is less than or equal to 65%, according to the SAM Penetration Test Method.

In a thirty-seventh embodiment, the method of any of the thirty-third through thirty-sixth embodiments may further comprise wherein a total amount of free superabsorbent particles within the absorbent structure is less than 0.5g, according to the Free SAM Determination Test Method.

What is claimed is:

1. An absorbent article extending in a longitudinal and a lateral direction and comprising:
an inner bodyside liner;
an outer cover; and
an absorbent structure disposed between the inner bodyside liner and the outer cover, the absorbent structure comprising:
a top facing material layer disposed adjacent the bodyside liner;
a bottom facing material layer disposed adjacent the outer cover;
a lofty nonwoven reinforcing material disposed between the top facing material layer and the bottom facing material layer; and
a first absorbent layer comprising superabsorbent particles intermixed with adhesive filaments disposed between the top facing material layer and the lofty nonwoven reinforcing material or between the lofty nonwoven reinforcing material and the bottom facing material layer,
wherein the superabsorbent particles are present in an amount greater than or equal to 90%, by weight of absorbent material between the top facing material layer and the bottom facing material layer, and
wherein the adhesive filaments form a three-dimensional mesh network comprising network adhesive filaments with the superabsorbent particles immobilized within the mesh network, the network adhesive filaments and superabsorbent particles extending throughout a three-dimensional space defined by the network adhesive filaments and the superabsorbent particles, with the network adhesive filaments extending in random orientations throughout the three-dimensional space.

2. The absorbent article of claim 1, wherein the absorbent structure comprises only the first absorbent layer disposed between the top facing material layer and the lofty nonwoven reinforcing material or between the lofty nonwoven reinforcing material and the bottom facing material layer and does not comprise a second absorbent layer disposed between the other of the top facing material layer and the lofty nonwoven reinforcing material or between the lofty nonwoven reinforcing material and the bottom facing material layer.

3. The absorbent article of claim 2, wherein the superabsorbent particles are disposed within the absorbent structure at an average basis weight of between 50 gsm and 600 gsm.

4. The absorbent article of claim 1, wherein the first absorbent layer is disposed between the top facing material layer and the lofty nonwoven reinforcing material, and wherein the absorbent structure further comprises a second absorbent layer disposed between the lofty nonwoven reinforcing material and the bottom facing material layer.

5. The absorbent article of claim 4, wherein the superabsorbent particles of the first absorbent layer are present in an amount between 100 gsm and 250 gsm, and wherein the superabsorbent particles of the second absorbent layer are present in an amount between 100 gsm and 250 gsm.

6. The absorbent article of claim 5, wherein the superabsorbent particles of the first absorbent layer are present in an amount measured in gsm that is greater than or equal to 75% of an amount of the superabsorbent particles of the second absorbent layer, also as measured in gsm.

7. The absorbent article of claim 4, wherein the adhesive filaments are present in an amount less than 5%, by weight of the superabsorbent particles.

8. The absorbent article of claim 4, wherein the adhesive filaments are present in an amount less than 4%, by weight of the superabsorbent particles.

9. The absorbent article of claim 3, wherein the adhesive filaments and the superabsorbent particles intermix prior to deposition onto a forming surface to form the absorbent structure, and wherein the absorbent structure lacks adhesive other than the adhesive intermixed with the superabsorbent particles.

10. The absorbent article of claim 1, wherein the absorbent garment lacks a surge or distribution layer between the absorbent structure and the bodyside liner.

11. The absorbent article of claim 1, wherein the absorbent structure is cellulose free.

12. An absorbent article extending in a longitudinal and a lateral direction and comprising:
an inner bodyside liner;
an outer cover; and
an absorbent structure disposed between the inner bodyside liner and the outer cover, the absorbent structure comprising:
a top facing material layer disposed adjacent the bodyside liner;
a bottom facing material layer disposed adjacent the outer cover;
a lofty nonwoven reinforcing material disposed between the top facing material layer and the bottom facing material layer;
a first absorbent layer comprising superabsorbent particles intermixed with adhesive filaments disposed between the top facing material layer and the lofty nonwoven reinforcing material, the superabsorbent particles of the first absorbent layer being present in an amount between 100 gsm and 250 gsm; and
a second absorbent layer comprising superabsorbent particles intermixed with adhesive filaments disposed between the lofty nonwoven reinforcing material the bottom facing material layer, the superabsorbent particles of the second absorbent layer being present in an amount between 100 gsm and 250 gsm,
wherein the superabsorbent particles are present in an amount greater than or equal to 90%, by weight of absorbent material between the top facing material layer and the bottom facing material layer,
wherein a total quantity of adhesive disposed between the top facing material layer and the bottom facing material layer is present in an amount less than 4.5%, by weight of the superabsorbent particles present between the top facing material layer and the bottom facing material layer, and
wherein the adhesive filaments form a three-dimensional mesh network comprising network adhesive filaments with the superabsorbent particles immobilized within the mesh network, the network adhesive filaments and superabsorbent particles extending throughout a three-dimensional space defined by the network adhesive filaments and the superabsorbent particles, with the network adhesive filaments extending in random orientations throughout the three-dimensional space.

13. The absorbent article of claim 12, wherein the superabsorbent particles of the first absorbent layer are present in an amount greater than 0 gsm and less than 200 gsm, the superabsorbent particles of the second absorbent layer are present in an amount greater than 0 gsm and less than 200 gsm.

14. The absorbent article of claim 13, wherein a total quantity of adhesive disposed between the top facing material layer and the bottom facing material layer is present in an amount less than 3.0%, by weight of the superabsorbent particles present between the top facing material layer and the bottom facing material layer, and wherein a SAM penetration value of the absorbent structure is less than or equal to 65%, according to the SAM Penetration Test Method.

15. The absorbent article of claim 13, wherein a total quantity of adhesive disposed between the top facing material layer and the bottom facing material layer is present in an amount less than 3.0%, by weight of the superabsorbent particles present between the top facing material layer and the bottom facing material layer, and wherein a total amount of free superabsorbent particles within the absorbent structure is less than 0.5g, according to the Free Sam Determination Test Method.

16. The absorbent article of claim 12, wherein the absorbent structure is cellulose free.

17. The absorbent article of claim 12, wherein the adhesive filaments and the superabsorbent particles intermix prior to deposition onto a forming surface to form the absorbent structure, and wherein the absorbent structure lacks adhesive other than the adhesive intermixed with the superabsorbent particles.

18. An absorbent article extending in a longitudinal and a lateral direction and comprising:
an inner bodyside liner;
an outer cover; and
an absorbent structure disposed between the inner bodyside liner and the outer cover comprising superabsorbent particles present in an amount greater than or equal to 90%, by weight of absorbent material of the absorbent structure, and further comprising:
a top facing material layer disposed adjacent the bodyside liner;
a bottom facing material layer disposed adjacent the outer cover;
a lofty nonwoven reinforcing material disposed between the top facing material layer and the bottom facing material layer;
a first absorbent layer comprising superabsorbent particles intermixed with adhesive filaments disposed between the top facing material layer and the lofty nonwoven reinforcing material, the superabsorbent particles of the first absorbent layer being present in an amount greater than 0 gsm and less than 200 gsm; and
a second absorbent layer comprising superabsorbent particles intermixed with adhesive filaments disposed between the lofty nonwoven reinforcing material the bottom facing material layer, the superabsorbent particles of the second absorbent layer being present in an amount greater than 0 gsm and less than 200 gsm,
wherein a total quantity of adhesive disposed between the top facing material layer and the bottom facing material layer is present in an amount less than 4.0%, by weight of the superabsorbent particles present between the top facing material layer and the bottom facing material layer,
wherein the adhesive filaments form a three-dimensional mesh network comprising network adhesive filaments with the superabsorbent particles immobilized within the mesh network, the network adhesive filaments and superabsorbent particles extending throughout a three-dimensional space defined by the network adhesive filaments and the superabsorbent particles, with the network adhesive filaments extending in random orientations throughout the three-dimensional space, and
wherein a SAM penetration value of the absorbent structure is less than or equal to 65%, according to the SAM Penetration Test Method, according to the SAM Penetration Test Method.

19. The absorbent article of claim 18, wherein a total quantity of adhesive disposed between the top facing material layer and the bottom facing material layer is present in an amount less than 3.0%, by weight of the superabsorbent particles present between the top facing material layer and the bottom facing material layer.

20. The absorbent article of claim 18, wherein a total amount of free superabsorbent particles within the absorbent structure is less than 0.5g, according to the Free Sam Determination Test Method.

21. The absorbent article of claim 18, wherein the absorbent structure is cellulose free.

22. The absorbent article of claim 18, wherein the adhesive filaments and the superabsorbent particles intermix prior to deposition onto a forming surface to form the absorbent structure, and wherein the absorbent structure lacks adhesive other than the adhesive intermixed with the superabsorbent particles.
